(19) United States
(12) Patent Application Publication
(10) Pub. No.: US 2007/0004810 A1
(43) Pub. Date: Jan. 4, 2007

(54) Novel catalyst and fischer-tropsch synthesis process using same

(76) Inventors: Yong Wang, Richland, WA (US); Chunshe Cao, Kennewick, WA (US); Rachid Taha, Dublin, OH (US); Rick B. Watson, Columbus, OH (US); David VanderWiel, Brecksville, OH (US); Anna Lee Tonkovich, Dublin, OH (US); Kai Tod Paul Jarosch, Bexley, OH (US)

Correspondence Address: Neil A. DuChez;Renner, Otto, Boisselle & Sklar, LLP, Nineteenth Floor, 1621 Euclid Avenue, Cleveland, OH, 44115, US

(21) Appl. No.: 11/170,907

(22) Filed: Jun. 30, 2005

(51) Int. Cl. C07C 27/06

(52) U.S. Cl. 518/718

(57) ABSTRACT

The disclosed invention relates to a novel cobalt catalyst and to a process for converting a reactant composition comprising H₂ and CO to a product comprising at least one aliphatic hydrocarbon having at least about 5 carbon atoms in a reactor using the catalyst. The process can be conducted in any reactor and is particularly suitable for microchannel reactors.

### TECHNICAL FIELD

This invention relates to a novel catalyst and a Fischer-Tropsch synthesis process using the catalyst. The process may be conducted in any reactor and is particularly suitable for microchannel reactors.

### BACKGROUND

The Fischer-Tropsch synthesis reaction involves converting a reactant composition comprising H₂ and CO in the presence of a catalyst to aliphatic hydrocarbon products. The reactant composition may comprise the product stream from another reaction process such as steam reforming (product stream H₂/CO˜3), partial oxidation (product stream H₂/CO˜2), autothermal reforming (product stream H₂/CO˜2.5), CO₂ reforming (H₂/CO˜1), coal gasification (product stream H₂/CO˜1), and combinations thereof. The aliphatic hydrocarbon products may range from methane to paraffinic waxes of up to 100 carbon atoms or more.

Conventional reactors such as tubular fixed bed reactors and slurry reactors have various problems in heat and mass transfer resulting in limitations of choice of process conditions for Fischer-Tropsch synthesis reactions. Hot spots in the fixed bed reactors significantly promote methane formation, reduce the heavy hydrocarbon selectivity and deactivate the catalyst. On the other hand, strong mass transfer resistance inherent in a catalyst suspended in a slurry system generally reduces the effective reaction rate and also causes difficulty in separation of catalysts from the products. This invention provides a solution to these problems.

### SUMMARY

This invention relates to a novel catalyst. The catalyst may be used in any Fischer-Tropsch synthesis reaction process in any reactor, including conventional slurry, fixed bed and fluidized bed processes. The catalyst is particularly suitable for Fischer-Tropsch synthesis reactions conducted in microchannel reactors. The inventive catalyst is a highly active catalyst which is particularly suitable for use in a microchannel reactor because the microchannel reactor provides enhanced heat transfer characteristics and more precise control of temperatures and residence times as compared to processes wherein microchannel reactors are not used. With at least one embodiment of this invention, it is possible to obtain relatively high levels of conversion of the CO, high levels of selectivity to the desired product (e.g., hydrocarbons in the middle distillate range), and relatively low levels of undesired products such as methane, by using the inventive catalyst in a microchannel reactor.

This invention relates to a catalyst, comprising: cobalt and optionally a co-catalyst and/or promoter on a support, the catalyst being made by the process comprising: (A) contacting the support with the cobalt and optionally the co-catalyst and/or promoter to form a supported catalyst; (C) oxidizing the supported catalyst; and (D) reducing the supported catalyst. In one embodiment, the process for making the catalyst further comprises subsequent to (A) put prior to (C) the following: (B) reducing the supported catalyst. In one embodiment, the process for making the catalyst further comprises subsequent to (D) the following: (E) oxidizing the supported catalyst; and (F) reducing the supported catalyst.

In one embodiment, the invention relates to a process for converting a reactant composition comprising H₂ and CO to a product comprising at least one aliphatic hydrocarbon having at least about 5 carbon atoms, the process comprising: flowing the reactant composition through a microchannel reactor in contact with the foregoing catalyst to convert the reactant composition to the product, the microchannel reactor comprising a plurality of process microchannels containing the catalyst; transferring heat from the process microchannels to a heat exchanger; and removing the product from the microchannel reactor.

### BRIEF DESCRIPTION OF THE DRAWINGS

In the annexed drawings, like parts and features have like designations.

FIG. 1 is a schematic illustration of a microchannel that may be used with the inventive process.

FIG. 2 is a schematic flow sheet illustrating the inventive Fischer-Tropsch synthesis process in a particular form wherein a reactant composition comprising CO and H₂ flows through a microchannel reactor in contact with the inventive catalyst and reacts to form a product comprising at least one aliphatic hydrocarbon.

FIG. 3 is a schematic illustration of a layer of process microchannels and a layer of heat exchange microchannels that may be used in the microchannel reactor core of the microchannel reactor illustrated in FIG. 2.

FIG. 4 is a schematic illustration showing the assembly of a microchannel reactor core that may be used in the microchannel reactor illustrated in FIG. 2.

FIG. 5 is a schematic illustration of a process microchannel and an adjacent heat exchange zone that may be used in the microchannel reactor core of the microchannel reactor illustrated in FIG. 2, the heat exchange zone containing a plurality of heat exchange channels extending lengthwise at right angles relative to the lengthwise direction of the process microchannel, the flow of heat exchange fluid through the heat exchange channels being cross-current relative to the flow of reactant composition and product through the process microchannel.

FIG. 6 is a schematic illustration of a process microchannel and an adjacent heat exchange channel that may be used in the microchannel reactor core of the microchannel reactor illustrated in FIG. 2, the flow of heat exchange fluid through the heat exchange channel being counter-current relative to the flow of reactant composition and product through the process microchannel.

FIG. 7 is a schematic illustration of a process microchannel and an adjacent heat exchange zone that may be used in the microchannel reactor core of the microchannel reactor illustrated in FIG. 2, the heat exchange zone containing a plurality of heat exchange channels extending lengthwise at right angles relative to the lengthwise direction of the process microchannel, the heat exchange zone extending lengthwise in the same direction as the process microchannel and being positioned at or near the process microchannel entrance, the length of the heat exchange zone being less than the length of the process microchannel.

FIG. 8 is a schematic illustration of a process microchannel and first and second adjacent heat exchange zones that may be used in the microchannel reactor core of the microchannel reactor illustrated in FIG. 2, each of the heat exchange zones containing a plurality of heat exchange channels extending lengthwise at right angles relative to the lengthwise direction of the process microchannel, the heat exchange zone extending lengthwise in the same direction as the process microchannel and being positioned at or near the process microchannel entrance, the length of the first heat exchange zone being less than the length of the process microchannel, the length of the second heat exchange zone being less than the length of the first heat exchange zone.

FIG. 9 is a schematic illustration of a process microchannel that may be used with the inventive process, the process microchannel containing a catalyst having a flow-by configuration.

FIG. 10 is a schematic illustration of a process microchannel that may be used with the inventive process, the process microchannel containing a catalyst having a flow-through configuration.

FIG. 11 is a schematic illustration of a process microchannel that may be used in the inventive process, the process microchannel containing a fin assembly comprising a plurality of fins, a catalyst being supported by the fins.

FIG. 12 illustrates an alternate embodiment of the process microchannel and fin assembly illustrated in FIG. 11.

FIG. 13 illustrates another alternate embodiment of the process microchannel and fin assembly illustrated in FIG. 11.

FIG. 14 is a plot showing results for the Fischer-Tropsch synthesis reaction described in Example 1.

FIG. 15 is a temperature programed reduction (TPR) plot for samples of Catalyst B described in Example 2.

FIG. 16 is a plot showing the relation between density and pore volume after each impregnation step for Catalyst C described in Example 3.

FIG. 17 is a plot showing the particle size distribution for Catalysts B and C described in Examples 2 and 3.

FIG. 18 is a TPR plot for Catalysts B and C from Examples 2 and 3.

FIG. 19 shows photo micrographs of Catalysts B and C from Examples 2 and 3.

### DETAILED DESCRIPTION

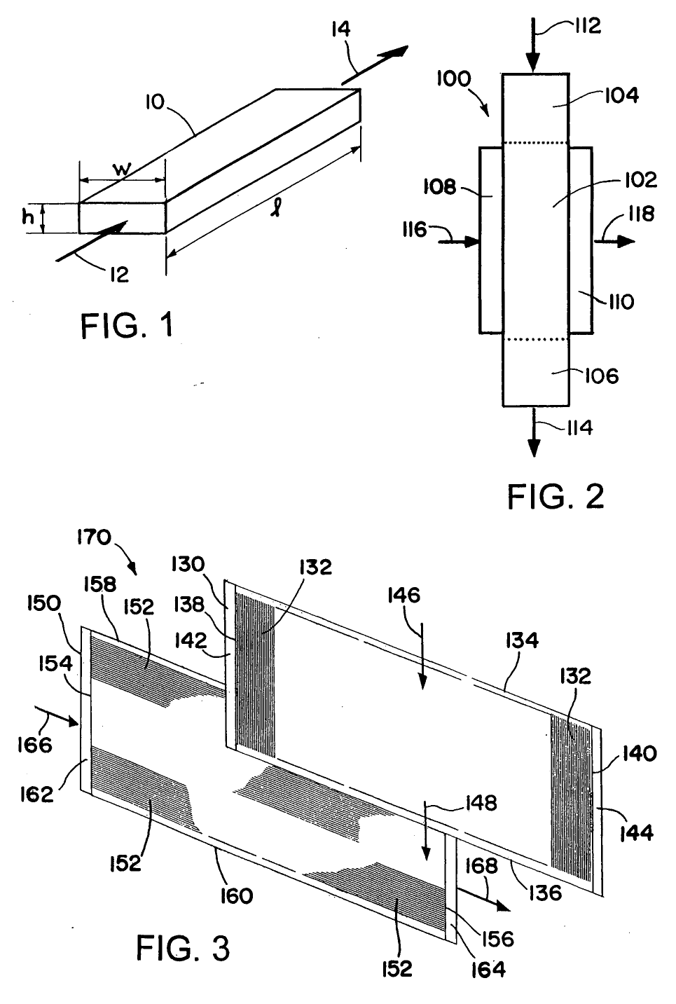

The term “microchannel” refers to a channel having at least one internal dimension of height or width of up to about 10 millimeters (mm), and in one embodiment up to about 5 mm, and in one embodiment up to about 2 mm, and in one embodiment up to about 1 mm. The flow of fluid through the microchannel may proceed along the length of the microchannel normal to the height and width of the microchannel. An example of a microchannel that may be used with the inventive process as a process microchannel and/or a heat exchange microchannel is illustrated in FIG. 1. The microchannel 10 illustrated in FIG. 1 has a height (h), width (w) and length (l). Fluid flows through the microchannel 10 along the length of the microchannel in the direction indicated by arrows 12 and 14. The height (h) or width (w) of the microchannel may be in the range of about 0.05 to about 10 mm, and in one embodiment about 0.05 to about 5 mm, and in one embodiment about 0.05 to about 2 mm, and in one embodiment about 0.05 to about 1.5 mm, and in one embodiment about 0.05 to about 1 mm, and in one embodiment about 0.05 to about 0.75 mm, and in one embodiment about 0.05 to about 0.5 mm. The other dimension of height or width may be of any dimension, for example, up to about 3 meters, and in one embodiment about 0.01 to about 3 meters, and in one embodiment about 0.1 to about 3 meters. The length (l) of the microchannel may be of any dimension, for example, up to about 10 meters, and in one embodiment about 0.2 to about 10 meters, and in one embodiment from about 0.2 to about 6 meters, and in one embodiment from 0.2 to about 3 meters. Although the microchannel 10 illustrated in FIG. 1 has a cross section that is rectangular, it is to be understood that the microchannel may have a cross section having any shape, for example, a square, circle, semi-circle, trapezoid, etc. The shape and/or size of the cross section of the microchannel may vary over its length. For example, the height or width may taper from a relatively large dimension to a relatively small dimension, or vice versa, over the length of the microchannel.

The term “adjacent” when referring to the position of one channel relative to the position of another channel means directly adjacent such that a wall separates the two channels. This wall may vary in thickness. However, “adjacent” channels are not separated by an intervening channel that would interfere with heat transfer between the channels. In one embodiment, one channel may be adjacent to another channel over only part of the dimension of the another channel. For example, a process microchannel may be longer than and extend beyond one or more adjacent heat exchange channels.

The term “fluid” refers to a gas, a liquid, a mixture of a gas and a liquid, or a gas or a liquid containing dispersed solids or liquid droplets.

The term “contact time” refers to the volume of the reaction zone within the microchannel reactor divided by the volumetric feed flow rate of the reactant composition at a temperature of 0 C and a pressure of one atmosphere.

The term “residence time” refers to the internal volume of a space (e.g., the reaction zone within a microchannel reactor) occupied by a fluid flowing through the space divided by the average volumetric flowrate for the fluid flowing through the space at the temperature and pressure being used.

The term “reaction zone” refers to the space within the process microchannels wherein the reactants contact the catalyst.

The term “conversion of CO” refers to the CO mole change between the reactant composition and product divided by the moles of CO in the reactant composition.

The term “selectivity to methane” refers to the moles of methane in the product divided by the moles of methane plus two times the number of moles of C₂ hydrocarbons in the product, plus three times the number of moles of C₃ hydrocarbons in the product, plus four times the number of moles of C₄ hydrocarbons in the product, etc., until all of the moles of hydrocarbons in the product have been included.

The term “one-pass conversion of CO” refers to the conversion of CO after one pass through the microchannel reactor employed with the inventive process. The term “yield of product” refers to conversion of CO multiplied by selectivity to the indicated product(s).

The term “metal dispersion” refers to the percent of catalytically active metal atoms and promoter atoms on the surface of the catalyst as compared to the total number of metal atoms in the catalyst as measured by hydrogen chemisorption which is described in “Heterogeneous Catalysis in Industrial Practice,” 2^{nd} ed., Charles N. Satterfield, p. 139, McGraw Hill (1996), which is incorporated herein by reference.

In the expression “about 0.5 gram of aliphatic hydrocarbon having at least about 5 carbon atoms per gram of catalyst per hour” the weight or number of grams of catalyst refers to the total weight of the catalyst comprising cobalt or an oxide thereof, optional co-catalyst (e.g., Re or Ru), and/or promoter (e.g., Na, K, etc.) as well as the weight of the support (e.g., alumina). However, if the catalyst is supported on an engineered support structure such as a foam, felt, wad or fin, the weight of such engineered support structure is not included in the calculation of the weight or number of grams of catalyst. Similarly, if the catalyst is adhered to the microchannel walls, the weight of the microchannel walls is not included in the calculation.

The term “Co loading” refers to the weight of the Co in the catalyst divided by the total weight of the catalyst, that is, the total weight of the Co plus any co-catalyst or promoter as well as the support. If the catalyst is supported on an engineered support structure such as a foam, felt, wad or fin, the weight of such engineered support structure is not included in the calculation. Similarly, if the catalyst is adhered to the microchannel walls, the weight of the microchannel walls is not included in the calculation.

Unless otherwise indicated, all pressures are expressed in terms of absolute pressure.

The inventive catalyst may comprise a supported catalyst, the active portion of the catalyst comprising at least one composition represented by the formula
COM¹ₐ M²_{b}Oₓ
wherein: M¹ may be Fe, Ni, Ru, Re, Os or a mixture thereof, and in one embodiment M¹ may be Ru or Re or a mixture thereof; M² may be Li, B, Na, K, Rb, Cs, Mg, Ca, Sr, Ba, Sc, Y, La, Ac, Zr, La, Ac, Ce, Th, or a mixture of two or more thereof, and, in one embodiment M² may be Na, K, or a mixture thereof; a may be a number in the range of zero to about 0.5, and in one embodiment zero to about 0.2; b may be a number in the range of zero to about 0.5, and in one embodiment zero to about 0.1; and x is the number of oxygens needed to fulfill the valency requirements of the elements present. M¹ may be referred to as a co-catalyst. M² may be referred to as a promoter.

The support material may comprise alumina, zirconia, silica, aluminum fluoride, fluorided alumina, bentonite, ceria, zinc oxide, silica-alumina, silicon carbide, or a combination of two or more thereof. The support material may comprise a refractory oxide or a molecular sieve. In one embodiment the support material is other than titania.

In one embodiment, the inventive catalyst may comprise cobalt supported on alumina. In one embodiment, the inventive catalyst may comprise cobalt and rhenium supported on alumina. In one embodiment, the inventive catalyst is other than a catalyst comprising cobalt and ruthenium supported on titania.

The inventive catalyst may be made using a process comprising multiple contacting steps with calcination steps conducted between each contacting step, followed by multiple reductions and oxidations. The contacting steps may be referred to as impregnating steps. The reduction and oxidation steps may be referred to as redox steps or cycles. The use of this process, at least in one embodiment, allows for the formation of catalysts with levels of loading of cobalt and, optionally, one or more co-catalysts and/or promoters that are higher as compared to processes wherein these procedures are not employed. The use of this process, at least in one embodiment, provides for a catalyst that is more active as compared to catalysts made by processes wherein these procedures are not used. In one embodiment, Co and optionally a co-catalyst (e.g., Re or Ru) and/or promoter (e.g., Na or K) are loaded on a support (e.g., Al₂O₃) using the following steps: (A-1) contacting the support with a composition comprising cobalt and optionally a co-catalyst and/or promoter to provide a supported catalyst; (A-2) calcining the supported catalyst formed in step (A-1) to form a calcined catalyst; (A-3) contacting the calcined catalyst formed in (A-2) with another composition comprising cobalt and optionally a co-catalyst and/or promoter to form an enhanced catalyst product; and (A-4) calcining the enhanced catalyst product formed in step (A-3) to provide an intermediate supported catalyst.

The cobalt and optional co-catalyst and/or promoter may be impregnated on the support using an incipient wetness impregnation process. A solution of cobalt nitrate may be used to impregnate the support with cobalt. Steps (A-3) and (A-4) may be repeated one or more additional times until the desired loading of cobalt, and optional co-catalyst and/or promoter, is achieved. The process may be continued until the cobalt achieves a loading level of about 10% by weight or more, and in one embodiment about 15% or more, and in one embodiment about 20% by weight or more, and in one embodiment about 25% by weight or more, and in one embodiment about 28% by weight or more, and in one embodiment about 30% by weight or more, and in one embodiment about 32% by weight or more, and in one embodiment about 35% by weight or more, and in one embodiment about 37% by weight or more, and in one embodiment about 40% by weight or more. The Co dispersion may be at least about 3%, and in one embodiment at least about 5%, and in one embodiment at least about 7%. The loading of co-catalyst (e.g., Re or Ru) may be in the range up to about 40% by weight, and in one embodiment up to about 20% by weight, and in one embodiment up to about 10% by weight, and in one embodiment from about 0.1 to about 10% by weight, and in one embodiment from about 1 to about 6% by weight. The loading of promoter (e.g., Na or K) may be up to about 20% by weight, and in one embodiment up to about 5% by weight. Each of the calcination steps may comprise heating the catalyst at a temperature in the range from about 100° C. to about 500° C., and in one embodiment about 100° C. to about 400° C., and in one embodiment from about 250 to about 350° C., for about 0.5 to about 100 hours, and in one embodiment about 0.5 to about 24 hours, and in one embodiment about 2 to about 3 hours. The temperature may be ramped to the calcination temperature at a rate of about 1-20° C./min. The calcination steps may be preceded by drying steps wherein the catalyst is dried at a temperature in the range from about 75° C. to about 200° C., and in one embodiment from about 75° C. to about 150° C., for about 0.5 to about 100 hours, and in one embodiment about 0.5 to about 24 hours. In one embodiment, the catalyst may be dried for about 12 hours at about 90° C. and then at about 110-120° C. for about 1-1.5 hours, the temperature being ramped from 90° C. to 110-120° C. at a rate of about 0.5-1° C./min.

The intermediate supported catalyst from (A) may be oxidized and reduced. The supported catalyst may be oxidized using oxidation step (C) and, optionally, oxidation step (E). The supported catalyst may be reduced using reduction step (D) and, optionally, reduction steps (B) and/or (F). In one embodiment, step (A) may be conducted prior to positioning the catalyst in the process microchannels, and steps (C) and (D) may be conducted after the catalyst is positioned in the process microchannels. In one embodiment, step (A) may be conducted prior to positioning the catalyst in the process microchannels, and steps (B), (C) and (D) may be conducted after the catalyst is positioned in the process microchannels. In one embodiment, step (A) may be conducted prior to positioning the catalyst in the process microchannels, and steps (C), (D), (E) and (F) may be conducted after the catalyst is positioned in the process microchannels. In one embodiment, steps (B) through (F) may be conducted with the catalyst positioned in the process microchannels of the microchannel reactor. In one embodiment, steps (A) through (E) may be conducted prior to positioning the catalyst in the process microchannels, and step (F) may be conducted after the catalyst is positioned in the process microchannels.

Reduction step (B) may involve process steps (B-1) to (B-5). Steps (B-1) to (B-4) involve contacting the supported catalyst with a reducing fluid. The reduction step (B) may comprise: (B-1) heating the supported catalyst from ambient temperature to a first temperature in the range from about 100° C. to about 500° C., and in one embodiment from about 100° C. to about 400° C., and in one embodiment from about 200° C. to about 300° C., and in one embodiment about 250° C., over a period of time in the range from about 0.5 to about 12 hours, and in one embodiment in the range from about 0.5 to about 10 hours, and in one embodiment in the range from about 1 to about 5 hours, and in one embodiment about 3.8 hours; (B-2) maintaining the supported catalyst at the first temperature for a period of time in the range from about 2 to about 25 hours, and in one embodiment for a period of time in the range from about 2 to about 20 hours, and in one embodiment about 8 hours; (B-3) heating the supported catalyst from (B-2) to a second temperature in the range from about 300° C. to about 600° C., and in one embodiment in the range from about 400° C., over a period of time in the range from about 0.5 to about 12 hours, and in one embodiment in the range from about 0.5 to about 10 hours, and in one embodiment in the range from about 1 to about 3 hours, and in one embodiment about 2.5 hours; (B-4) maintaining the supported catalyst at the second temperature for a period of time in the range from about 0.5 to about 24 hours, and in one embodiment from about 2 to about 24 hours, and in one embodiment for a period of time in the range from about 5 to about 20 hours, and ftlinein one embodiment about 12 hours; and (B-5) contacting the supported catalyst from (B-4) with an inert fluid and cooling the supported catalyst to ambient temperature over a period of time in the range from about 1 to about 48 hours, and in one embodiment in the range from about 5 to about 48 hours, and in one embodiment about 13.2 hours. Steps (B-1) to (B-4) may be conducted at a pressure in the range from about 0.7 to about 20 atm, and in one embodiment about 1.25 to about 5 atm.

The oxidation step (C) may involve process steps (C-1) to (C-3). Steps (C-1) and (C-2) involve contacting the supported catalyst with an oxidizing fluid. The oxidation step (C) may comprise: (C-1) heating the supported catalyst from ambient temperature to a first temperature in the range from about 150° C. to about 650° C., and in one embodiment from about 200° C. to about 500° C., and in one embodiment about 350° C., over a period of time in the range from about 0.5 to about 12 hours, and in one embodiment in the range from about 1 to about 6 hours, and in one embodiment about 3.6 hours; (C-2) maintaining the supported catalyst at the first temperature for a period of time in the range from about 0.5 to about 10 hours, and in one embodiment for a period of time in the range from about 1 to about 3 hours, and in one embodiment about 2 hours; and (C-3) contacting the supported catalyst with an inert fluid and cooling the supported catalyst to ambient temperature over a period of time in the range from about 1 to about 48 hours, and in one embodiment in the range from about 2 to about 48 hours, and in one embodiment in the range from about 10 to about 30 hours, and in one embodiment about 18.1 hours. Steps (C-1) to (C-3) may be conducted at a pressure in the range from about 0.7 to about 20 atm, and in one embodiment about 1.25 to about 5 atm.

The reduction step (D) may involve process steps (D-1) to (D-5). Steps (D-1) to (D-4) involve contacting the supported catalyst with a reducing fluid. The reduction step (D) may comprise: (D-1) heating the supported catalyst to a first temperature in the range from about 100° C. to about 500° C., and in one embodiment from about 200° C. to about 300° C., and in one embodiment about 250° C., over a period of time in the range from about 0.5 to about 12 hours, and in one embodiment in the range from about 0.5 to about 10 hours, and in one embodiment in the range from about 1 to about 8 hours, and in one embodiment about 3.8 hours; (D-2) maintaining the supported catalyst at the first temperature for a period of time in the range from about 2 to about 25 hours, and in one embodiment in the range from about 5 to about 25 hours, and in one embodiment in the range from about 5 to about 20 hours, and in one embodiment about 8 hours; (D-3) heating the supported catalyst from (D-2) to a second temperature in the range from about 300° C. to about 600° C., and in one embodiment from about 350° C. to about 500° C., and in one embodiment about 400° C., over a period of time in the range from about 0.5 to about 12 hours, and in one embodiment in the range from about 0.5 to about 10 hours, and in one embodiment from about 1 to about 3 hours, and in one embodiment about 2.5 hours; (D-4) maintaining the supported catalyst at the second temperature for a period of time in the range from about 0.5 to about 24 hours, and in one embodiment in the range from about 5 to about 20 hours, and in one embodiment about 12 hours; and (D-5) contacting the supported catalyst with an inert fluid and cooling the supported catalyst to ambient temperature over a period of time in the range from about 1 to about 48 hours, and in one embodiment in the range from about 5 to about 48 hours, and in one embodiment in the range from about 5 to about 20 hours, and in one embodiment about 24 hours. Steps (D-1) to (D-5) may be conducted at a pressure in the range from about 0.7 to about 20 atm; and in one embodiment about 1.25 to about 5 atm.

The oxidation step (E) may involve process steps (E-1) to (E-3). Steps (E-1) and (E-2) involve contacting the supported catalyst with an oxidizing fluid. The oxidation step (E) may comprise: (E-1) heating the supported catalyst to a first temperature in the range from about 150° C. to about 650° C., and in one embodiment from about 250° C. to about 450° C., and in one embodiment about 350° C., over a period of time in the range from about 0.5 to about 12 hours, and in one embodiment in the range from about 1 to about 6 hours, and in one embodiment about 3.6 hours; (E-2) maintaining the supported catalyst at the first temperature for a period of time in the range from about 0.5 to about 7 hours, and in one embodiment in the range from about 1 to about 3 hours, and in one embodiment about 2 hours; and (E-3) contacting the supported catalyst with an inert fluid and cooling the supported catalyst to ambient temperature over a period of time in the range from about 1 to about 48 hours, and in one embodiment in the range from about 2 to about 48 hours, and in one embodiment in the range from about 5 to about 30 hours, and in one embodiment about 22 hours. Steps (E-1) to (E-3) may be conducted at a pressure in the range from about 0.7 to about 20 atm, and in one embodiment from about 1.25 to about 5 atm.

The reduction step (F) may involve process steps (F-1) to (F-5). These steps involve contacting the supported catalyst with a reducing fluid. The reduction step (F) may comprise: (F-1) heating the supported catalyst to a first temperature in the range from about 100° C. to about 500° C., and in one embodiment from about 100° C. to about 400° C., and in one embodiment about 250° C., over a period of time in the range from about 0.5 to about 20 hours, and in on embodiment in the range from about 0.5 to about 12 hours, and in one embodiment about 3.8 hours; (F-2) maintaining the supported catalyst at the first temperature for a period of time in the range from about 0.5 to about 48 hours, and in one embodiment in the range from about 2 to about 20 hours, and in one embodiment about 12 hours, and in one embodiment about 1 hour; (F-3) heating the supported catalyst from (F-2) to a second temperature in the range from about 100° C. to about 600° C., and in one embodiment from about 300° C. to about 600° C., and in one embodiment about 375° C., over a period time in the range from about 0.5 to about 12 hours, and in one embodiment in the range from about 1 to about 5 hours; and in one embodiment about 2.1 hours; (F-4) maintaining the supported catalyst at the second temperature for about 0.5 to about 48 hours, and in one embodiment in the range from about 2 to about 24 hours, and in one embodiment about 12 hours; and (F-5) contacting the supported catalyst with a reducing fluid at a pressure in the range from about 0.7 to about 75 atm, and in one embodiment in the range from about 10 to about 50 atm, and in one embodiment about 28.2 atm, and cooling the supported catalyst to a temperature in the range from about 60° C. to about 250° C., and in one embodiment from about 100° C. to about 230° C., and in one embodiment about 160° C., over a period of time in the range from about 0.5 to about 40 hours, and in one embodiment in the range from about 1 to about 20 hours. Steps (F-1) to (F-4) may be conducted at a pressure in the range from about 0.7 to about 20 atm, and in one embodiment from about 1.25 to about 5 atm.

The process steps used in reduction steps (B), (D) and (F), which involve contacting the supported catalyst with a reducing fluid, may involve using hydrogen, hydrazine, or a mixture thereof as the reducing medium. A gaseous mixture comprising hydrogen and at least one inert gas may be used. The inert gas may be nitrogen, helium or argon. The concentration of hydrogen in the gaseous mixture may be in the range from about 0.25 to about 99.75% by volume, and in one embodiment in the range from about 0.25 to about 50%, and in one embodiment in the range from about 0.25 to about 20%, and in one embodiment in the range from about 1 to about 10% by volume, and in one embodiment about 5% by volume.

The process steps used in oxidation steps (C) and (E), which involve contacting the supported catalyst with an oxidizing fluid, may involve using oxygen, a peroxide (e.g., hydrogen peroxide) or a mixture thereof. A gaseous mixture comprising oxygen and at least one inert gas may be used. The inert gas may be nitrogen, helium or argon. The concentration of oxygen in the gaseous mixture may be in the range from about 0.25 to about 100% by volume, and in one embodiment in the range from about 0.25 to about 50%, and in one embodiment in the range from about 0.25 to about 20%, and in one embodiment in the range from about 0.5 to about 10% by volume, and in one embodiment about 2% by volume.

The inert fluid used in steps (B-5), (C-3), (D-5) and (E-3) may be an inert gas. The inert gas may comprise nitrogen, helium, argon, or a mixture of two or more thereof.

Referring to FIG. 2, the process for converting H₂ and CO to hydrocarbons may be conducted using microchannel reactor 100 which includes microchannel reactor core 102, reactant header 104, product footer 106, heat exchange header 108 and heat exchange footer 110. The microchannel reactor core 102 contains a plurality of process microchannels and a plurality of heat exchange channels adjacent to the process microchannels. The heat exchange channels may be microchannels. The process microchannels and heat exchange channels may be aligned in layers, one above the other, or side by side. A Fischer-Tropsch catalyst is contained within the process microchannels. The process header 104 provides a passageway for fluid to flow into the process microchannels with an even or substantially even distribution of flow to the process microchannels. The process footer 106 provides a passageway for fluid to flow from the process microchannels in a rapid manner with a relatively high rate of flow. The reactant composition flows into the microchannel reactor 100 through the reactant header 104, as indicated by directional arrow 112. The reactant composition may be preheated prior to entering the reactant header 104. The reactant composition flows through the process microchannels in the microchannel reactor core 102 in contact with the catalyst and reacts to form the desired product. In one embodiment, the flow of reactant composition and product through the reactor core 102 is in a vertical direction, from the top of the reactor core 102 to its bottom. The product, and in one embodiment unreacted components from the reactant composition, flow from the reactor core 102 through the product footer 106, and out of product footer 106, as indicated by directional arrow 114. Although an advantage of the inventive process is that a high level of conversion of CO may be obtained with one pass through the process microchannels, in one embodiment, unreacted components from the reactant composition or a portion thereof may be recycled back through the process microchannels in contact with the catalyst. The unreacted components of the reactant composition being recycled through the process microchannels may be recycled any number of times, for example, one, two, three, four times, etc. A heat exchange fluid flows into heat exchange header 108, as indicated by directional arrow 116, and from heat exchange header 108 through the heat exchange channels in microchannel reactor core 102 to heat exchange footer 110, and out of heat exchange footer 110, as indicated by directional arrow 118. The microchannel reactor 100 is employed in conjunction with storage vessels, pumps, valves, flow control devices, and the like, which are not shown in the drawings, but would be apparent to those skilled in the art.

In one embodiment, the microchannel reactor core 102 may contain layers of process microchannels and heat exchange microchannels aligned side by side. An example of such microchannels layers is illustrated in FIG. 3. Referring to FIG. 3, process microchannel layers 130 and heat exchange microchannel layers 150 are stacked side by side to provide repeating unit 170. Microchannel layer 130 provides for the flow of reactant and product. Microchannel layer 150 provides for the flow of heat exchange fluid.

Microchannel layer 130 contains a plurality of microchannels 132 aligned in parallel, each process microchannel 132 extending in a vertical direction along the length of microchannel layer 130 from end 134 to end 136, the process microchannels 132 extending along the width of microchannel layer 130 from end 138 to end 140. Bonding strips 142 and 144 are positioned at the ends 138 and 140, respectively, of microchannel layer 130 to permit bonding of the microchannel layer 130 to the next adjacent heat exchange layers 150. A catalyst is contained within the process microchannels 132. The flow of reactant and product through the process microchannels 132 may be in the direction indicated by arrows 146 and 148. Each of the process microchannels 132 may have a cross section having any shape, for example, a square, rectangle, circle, semi-circle, etc. The internal height of each process microchannel 132 may be considered to be the vertical or horizontal distance or gap between the microchannel layer 130 and the next adjacent heat exchange layer 150. Each process microchannel 132 may have an internal height of up to about 10 mm, and in one embodiment up to about 6 mm, and in one embodiment up to about 4 mm, and in one embodiment up to about 2 mm. In one embodiment, the height may be in the range of about 0.05 to about 10 mm, and in one embodiment about 0.05 to about 6 mm, and in one embodiment about 0.05 to about 4 mm, and in one embodiment about 0.05 to about 2 mm. The width of each of these microchannels may be of any dimension, for example, up to about 3 meters, and in one embodiment about 0.01 to about 3 meters, and in one embodiment about 0.1 to about 3 meters. The length of each process microchannel 132 may be of any dimension, for example, up to about 10 meters, and in one embodiment about 0.2 to about 10 meters, and in one embodiment from about 0.2 to about 6 meters, and in one embodiment from 0.2 to about 3 meters.

Microchannel layer 150 contains a plurality of heat exchange microchannels 152 aligned in parallel, each heat exchange microchannel 152 extending horizontally along the width of microchannel layer 150 from end 154 to end 156, the heat exchange microchannels 152 extending along the length of microchannel layer 150 from end 158 to end 160 of microchannel layer 150. Bonding strips 162 and 164 are positioned at ends 154 and 156, respectively, of microchannel layer 150 to permit bonding of the microchannel layer 150 to the next adjacent process microchannel layers 130. The heat exchange fluid may flow through the heat exchange microchannels 152 in the direction indicated by arrows 166 and 168. The flow of heat exchange fluid in the direction indicated by arrows 166 and 168 is cross-current to the flow of reactant and product flowing through process microchannels 132 as indicated by arrows 146 and 148. Alternatively, the heat exchange microchannels 152 could be oriented to provide for flow of the heat exchange fluid along the width of the microchannel layer 150 from end 158 to end 160 or from end 160 to end 158. This would result in the flow of heat exchange fluid in a direction that would be cocurrent or counter-current to the flow of reactant and product through the process microchannels 132. Each of the heat exchange microchannels 152 may have a cross section having any shape, for example, a square, rectangle, circle, semi-circle, etc. The internal height of each heat exchange microchannel 152 may be considered to be the vertical or horizontal distance or gap between the heat exchange microchannel layer 150 and the next adjacent microchannel layer 130. Each of the heat exchange microchannels 152 may have an internal height of up to about 2 mm, and in one embodiment in the range of about 0.05 to about 2 mm, and in one embodiment about 0.05 to about 1.5 mm. The width of each of these microchannels may be of any dimension, for example, up to about 3 meters, and in one embodiment from about 0.01 to about 3 meters, and in one embodiment about 0.1 to about 3 meters. The length of each of the heat exchange microchannels 152 may be of any dimension, for example, up to about 10 meters, and in one embodiment from about 0.2 to about 10 meters, and in one embodiment from about 0.2 to about 6 meters, and in one embodiment from 0.2 to about 3 meters.

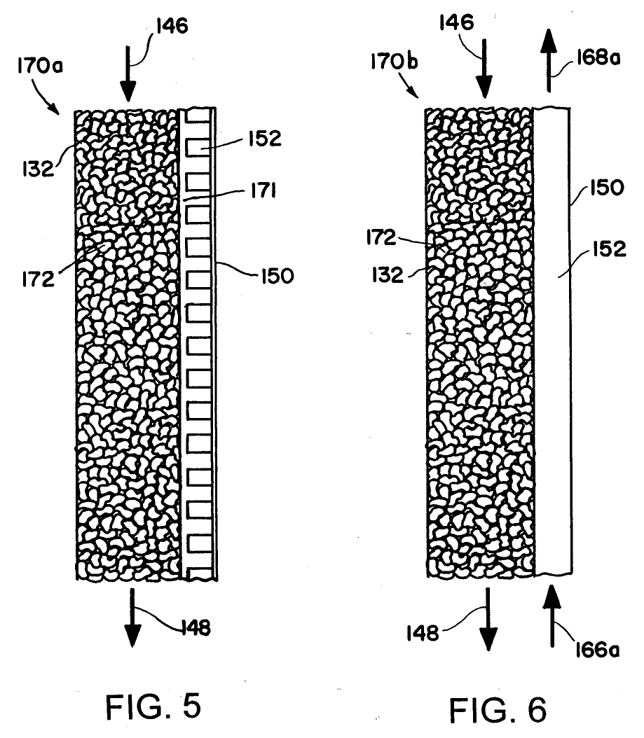

The process microchannels and heat exchange microchannels may be aligned as provided in repeating unit 170a. Repeating unit 170a is illustrated in FIG. 5. Referring to FIG. 5, process microchannel 132 is positioned adjacent to microchannel layer 150 which contains heat exchange microchannels 152. A common wall 171 separates the process microchannel 132 from the heat exchange microchannel layer 150. A catalyst 172 is packed into the process microchannel 132. The reactant composition flows into and through the packed bed of catalyst 172 in process microchannel 132 in the direction indicated by directional arrow 146, contacts catalyst 172 and reacts to form the desired product. The product, and in one embodiment unreacted components from the reactant composition, exit the process microchannel 132 as indicated by directional arrow 148. Heat exchange fluid flows through the heat exchange microchannels 152 in a direction that is cross-current to the flow of reactant composition and product through the process microchannel 132.

The process microchannels and heat exchange microchannels may be aligned as provided in repeating unit 170b. Repeating unit 170b illustrated in FIG. 6 is identical to the repeating unit 170a illustrated in FIG. 5 with the exception that the microchannel layer 150 is rotated 90° and the heat exchange fluid flowing through the heat exchange microchannel 152 flows in the direction indicated by direction arrows 166a and 168a which is countercurrent to the flow of reactant composition and product through the process microchannel 132. Alternatively, the heat exchange fluid could flow in the direction opposite to that indicated by directional arrows 166a and 168a and thereby provide for the flow of heat exchange fluid through the heat exchange microchannel 152 in a direction that would be cocurrent relative to the direction of reactant composition and product through the process microchannel 132.

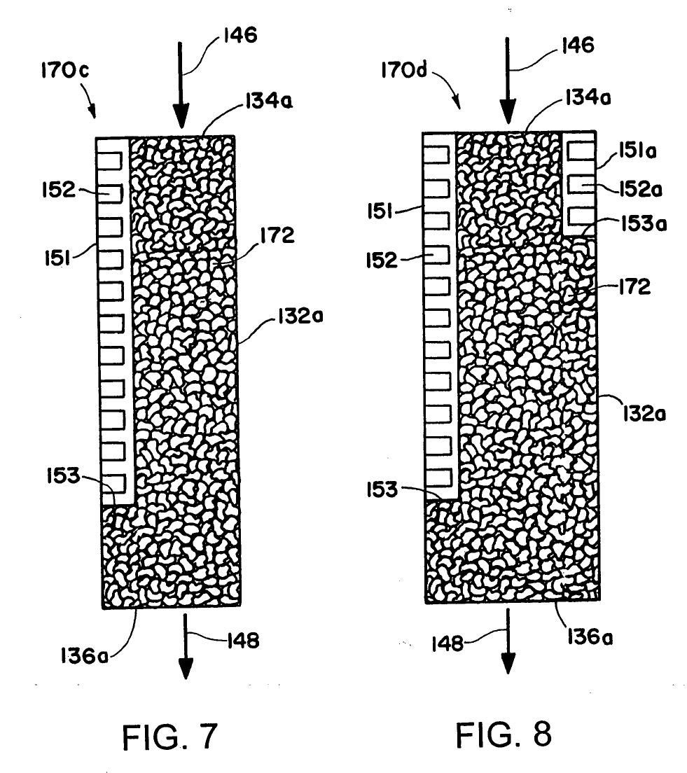

The process microchannels and heat exchange microchannels may be aligned as provided in repeating unit 170c. Repeating unit 170c is illustrated in FIG. 7. Referring to FIG. 7, process microchannel 132a is positioned adjacent to heat exchange zone 151. Heat exchange zone 151 contains a plurality of heat exchange microchannels 152 aligned in parallel relative to one another, each heat exchange microchannel 152 extending lengthwise at a right angle relative to the lengthwise direction of the process microchannel 132a. Heat exchange zone 151 is shorter in length than process microchannel 132a. Heat exchange zone 151 extends lengthwise from or near the entrance 134a to process microchannel 132a to a point along the length of the process microchannel 132a short of the exit 136a to the process microchannel 132a. In one embodiment, the length of heat exchange zone 151 is up to about 100% of the length of process microchannel 132a, and in one embodiment the length of heat exchange zone 151 is from about 5 to about 100% of the length of the process microchannel 132a, and in one embodiment the length of the heat exchange zone 151 is from about 5 to about 50% of the length of the process microchannel 132a, and in one embodiment the length of the heat exchange zone 151 is from about 50% to about 100% of the length of the process microchannel 132a. The width of the process microchannel 132a is expanded or extended in the area downstream of the end 153 of the heat exchange zone 151. This arrangement provides the advantage of heat exchange (i.e., cooling) at or near the entrance 134a to the process microchannel 132a as well as to parts of the process microchannel 132a downstream from the entrance. A catalyst 172 is packed in the process microchannel 132a. The reactant composition flows into and through the packed bed of catalyst 172 in process microchannel 132a in the direction indicated by directional arrow 146, contacts catalyst 172 and reacts to form the desired product. The product, and in one embodiment unreacted components from the reactant composition, exit the process microchannel 132a, as indicated by directional arrow 148. Heat exchange fluid flows through the heat exchange microchannels 152 in a direction that is cross-current to the flow of reactant composition and product through the process microchannel 132a.

The process microchannels and heat exchange microchannels may be aligned as provided in repeating unit 170d. Repeating unit 170d, which is illustrated in FIG. 8, is identical to the repeating unit 170c illustrated in FIG. 7 with the exception that repeating unit 170d includes heat exchange zone 151a adjacent to process microchannel 132a on the opposite side of the process microchannel 132a. from the heat exchange zone 151. Heat exchange zone 151a contains a plurality of parallel heat exchange microchannels 152a which are the same as or similar in size and design to the heat exchange microchannels 152 discussed above. Heat exchange zone 151a extends lengthwise from or near the entrance 134a to process microchannel 132a to a point along the length of process microchannel 132a short of the end 153 of heat exchange zone 151. The length of the heat exchange zone 151a may be shorter than the length of the heat exchange zone 151. In one embodiment, the length of the heat exchange zone 151a may be up to about 100% of the length of the process microchannel 132a, and in one embodiment the length of the heat exchange zone 151a is from about 5 to about 100% of the length of the process microchannel 132a, and in one embodiment the length of the heat exchange zone 151a is from about 5 to about 50% of the length of the process microchannel 132a, and in one embodiment the length of the heat exchange zone 151a is from about 50% to about 100% of the length of the process microchannel 132a. The width of the process microchannel 132a is expanded in the areas downstream of the ends 153 and 153a of the heat exchange zones 151 and 151a, respectively. This arrangement provides the advantage of heat exchange (i.e., cooling) at or near the entrance 134a to the process microchannel 132a as well to parts of the process microchannel 132a downstream from the entrance 134a. The use of the two heat exchange zones 151 and 151a allows for a relatively high level of heat exchange in the area of the process microchannel 132a near its entrance, and a relatively moderate heat exchange in the process microchannel downstream from about the end 153a of heat exchange zone 151a. Catalyst 172 is packed into the process microchannel 132a. The reactant composition flows into and through the packed bed of catalyst 172 in process microchannel 132a in the direction indicated by directional arrow 146, contacts the catalyst 172 and reacts to form the desired product. The product, and in one embodiment unreacted components from the reactant composition, exit the process microchannel 132a, as indicated by directional arrow 148. Heat exchange fluid flows through the heat exchange channels 151 and 151a in a direction which is cross-current to the flow of reactant composition and product through the process microchannel 132a.

The catalyst bed may be segregated into separate reaction zones in the process microchannels in the direction of flow through the process microchannels. In each reaction zone the length of one or more adjacent heat exchange zone(s) may vary in their dimensions. For example, in one embodiment, the length of the one or more adjacent heat exchange zones may be less than about 50% of the length of each reaction zone. Alternatively, the one or more heat exchange zones may have lengths that are more than about 50% of the length of each reaction zone up to about 100% of the length of each reaction zone.

The number of microchannels in each of the microchannel layers 130 and 150 may be any desired number, for example, one, two, three, four, five, six, eight, ten, hundreds, thousands, tens of thousands, hundreds of thousands, millions, etc. Similarly, the number of repeating units 170 (or 170a through 170d) of microchannel layers in the microchannel reactor core 102 may be any desired number, for example, one, two, three, four, six, eight, ten, hundreds, thousands, etc.

The microchannel reactor 100, including the microchannel reactor core 102, may be constructed of any material that provides sufficient strength, dimensional stability and heat transfer characteristics for carrying out the inventive process. Examples of suitable materials include steel (e.g., stainless steel, carbon steel, and the like), aluminum, titanium, nickel, and alloys of any of the foregoing metals, plastics (e.g., epoxy resins, UV cured resins, thermosetting resins, and the like), monel, inconel, ceramics, glass, composites, quartz, silicon, or a combination of two or more thereof. The microchannel reactor may be fabricated using known techniques including wire electrodischarge machining, conventional machining, laser cutting, photochemical machining, electrochemical machining, molding, water jet, stamping, etching (for example, chemical, photochemical or plasma etching) and combinations thereof. The microchannel reactor may be constructed by forming layers or sheets with portions removed that allow flow passage. A stack of sheets may be assembled via diffusion bonding, laser welding, diffusion brazing, and similar methods to form an integrated device. The microchannel reactor has appropriate manifolds, valves, conduit lines, etc. to control flow of the reactant composition and product, and flow of the heat exchange fluid. These are not shown in the drawings, but can be readily provided by those skilled in the art.

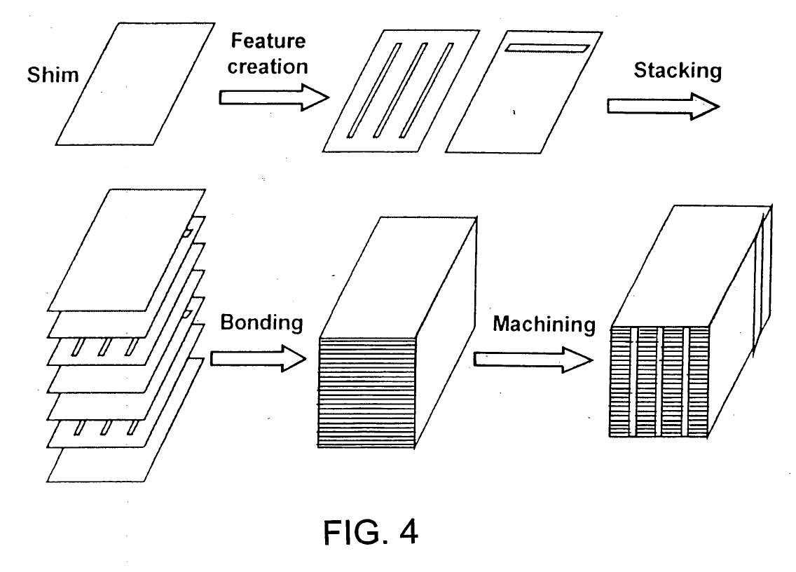

In one embodiment, the microchannel reactor 100 may be made by the process illustrated in FIG. 4. This process includes laminating or diffusion bonding thin sheets of any of the above-indicated materials (e.g., metal, plastic or ceramic) so that each layer has a defined geometry of channels and openings through which to convey fluids. After the individual layers are created, they may be stacked in a prescribed order to build up the lamination. The layers may be stacked side-by-side or one above the other. The completed stack may then be diffusion bonded to prevent fluids from leaking into or out of the microchannel reactor or between streams. After bonding, the device may be trimmed to its final size and prepared for attachment of pipes and manifolds. An additional step for the process microchannels that will contain the catalyst material is to integrate the catalyst into the device prior to final assembly.

Feature creation methods include photochemical etching, milling, drilling, electrical discharge machining, laser cutting, and stamping. A useful method for mass manufacturing is stamping. In stamping, care should be taken to minimize distortion of the material and maintain tight tolerances of channel geometries. Preventing distortion, maintaining shim alignment and ensuring that layers are stacked in the proper order are factors that should be controlled during the stacking process.

The stack may be bonded through a diffusion process. In this process, the stack is subjected to elevated temperatures and pressures for a precise time period to achieve the desired bond quality. Selection of these parameters may require modeling and experimental validation to find bonding conditions that enable sufficient grain growth between metal layers.

The next step, after bonding, is typically to machine the device. A number of processes may be used, including conventional milling with high-speed cutters, as well as highly modified electrical discharge machining techniques. A full-sized bonded microchannel reactor unit or sub-unit that has undergone post-bonding machining operations may comprise, for example, tens, hundreds or thousands of shims.

The reactant composition may comprise a mixture containing H₂ and CO. This mixture may be referred to as synthesis gas or syngas. The molar ratio of H₂ to CO may range from about 0.8 to about 10, and in one embodiment about 0.8 to about 5, and in one embodiment about 1 to about 3, and in one embodiment about 1.5 to about 3, and in one embodiment about 1.8 to about 2.5, and in one embodiment about 1.9 to about 2.2, and in one embodiment about 2.05 to about 2.10. The reactant composition may also contain CO₂ and/or H₂O, as well as light hydrocarbons of 1 to about 4 carbon atoms, and in one embodiment 1 to about 2 carbon atoms. The reactant composition may contain from about 5 to about 45% by volume CO, and in one embodiment about 5 to about 20% by volume CO; and about 55 to about 95% by volume H₂, and in one embodiment about 80 to about 95% by volume H₂. The concentration of CO₂ in the reactant composition may be up to about 60% by volume, and in one embodiment about 5 to about 60% by volume, and in one embodiment about 5 to about 40% by volume. The concentration of H₂O in the reactant composition may be up to about 80% by volume, and in one embodiment about 5 to about 80% by volume, and in one embodiment about 5 to about 50% by volume. The concentration of light hydrocarbons in the reactant composition may be up to about 50% by volume, and in one embodiment about 1 to about 50% by volume, and in one embodiment about 1 to about 50% by volume. The reactant composition may comprise recycled gaseous products formed during the inventive process. The reactant composition may comprise a stream (e.g., a gaseous stream) from another process such as a steam reforming process (product stream with H₂/CO mole ratio of about 3), a partial oxidation process (product stream with H₂/CO mole ration of about 2), an autothermal reforming process (product stream with H₂/CO mole ratio of about 2.5), a CO₂ reforming process (product stream with H₂/CO mole ratio of about 1), a coal gassification process (product stream with H₂/CO mole ratio of about 1), and combinations thereof.

The presence of contaminants such as sulfur, nitrogen, halogen, selenium, phosphorus, arsenic, and the like, in the reactant composition may be undesirable. Thus, in one embodiment of the invention, the foregoing contaminants may be removed from the reactant composition or have their concentrations reduced prior to conducting the inventive process. Techniques for removing these contaminants are well known to those of skill in the art. For example, ZnO guardbeds may be used for removing sulfur impurities. In one embodiment, the contaminant level in the reactant composition may be at a level of up to about 5% by volume, and in one embodiment up to about 1% by volume, and in one embodiment up to about 0.1% by volume, and in one embodiment up to about 0.05% by volume.

The heat exchange fluid may be any fluid. These include air, steam, liquid water, gaseous nitrogen, other gases including inert gases, carbon monoxide, molten salt, oils such as mineral oil, and heat exchange fluids such as Dowtherm A and Therminol which are available from Dow-Union Carbide.

The heat exchange fluid may comprise a stream of the reactant composition. This can provide process pre-heat and increase in overall thermal efficiency of the process.

In one embodiment, the heat exchange channels comprise process channels wherein an endothermic process is conducted. These heat exchange process channels may be microchannels. Examples of endothermic processes that may be conducted in the heat exchange channels include steam reforming and dehydrogenation reactions. Steam reforming of an alcohol that occurs at a temperature in the range of about 200° C. to about 300° C. is another example of such an endothermic process. The incorporation of a simultaneous endothermic reaction to provide an improved heat sink may enable a typical heat flux of roughly an order of magnitude above the convective cooling heat flux. The use of simultaneous exothermic and endothermic reactions to exchange heat in a microchannel reactor is disclosed in U.S. patent application Ser. No. 10/222,196, filed Aug. 15, 2002, which is incorporated herein by reference.

In one embodiment, the heat exchange fluid undergoes a partial or full phase change as it flows through the heat exchange channels. This phase change provides additional heat removal from the process microchannels beyond that provided by convective cooling. For a liquid heat exchange fluid being vaporized, the additional heat being transferred from the process microchannels would result from the latent heat of vaporization required by the heat exchange fluid. An example of such a phase change would be an oil or water that undergoes boiling. In one embodiment, about 50% by weight of the heat exchange fluid is vaporized.

The heat flux for convective heat exchange in the microchannel reactor may range from about 1 to about 25 watts per square centimeter of surface area of the process microchannels (W/cm²) in the microchannel reactor, and in one embodiment from about 1 to about 10 W/cm². The heat flux for phase change or simultaneous endothermic reaction heat exchange may range from about 1 to about 250 W/cm², and in one embodiment from about 1 to about 100 W/cm², and in one embodiment from about 1 to about 50 W/cm², and in one embodiment from about 1 to about 25 W/cm², and in one embodiment from about 1 to about 10 W/cm².

The cooling of the process microchannels during the inventive process, in one embodiment, is advantageous for controlling selectivity towards the main or desired product due to the fact that such added cooling reduces or eliminates the formation of undesired by-products from undesired parallel reactions with higher activation energies. As a result of this cooling, in one embodiment, the temperature of the reactant composition at the entrance to the process microchannels may be within about 200° C., and in one embodiment within about 150° C., and in one embodiment within about 100° C., and in one embodiment within about 50° C., and in one embodiment within about 25° C., and in one embodiment within about 10° C., of the temperature of the product (or mixture of product and unreacted reactants) at the exit of the process microchannels.

The catalyst used in a microchannel reactor may have any size and geometric configuration that fits within the process microchannels. The catalyst may be in the form of particulate solids (e.g., pellets, powder, fibers, and the like) having a median particle diameter of about 1 to about 1000 μm (microns), and in one embodiment about 10 to about 500 μm, and in one embodiment about 25 to about 250 μm. In one embodiment, the catalyst is in the form of a fixed bed of particulate solids.

In one embodiment, the catalyst is in the form of a fixed bed of particulate solids, the median particle diameter of the catalyst particulate solids is relatively small, and the length of each process microchannel is relatively short. The median particle diameter may be in the range of about 1 to about 1000 μm, and in one embodiment about 10 to about 500 μm, and the length of each process microchannel may be in the range of up to about 500 cm, and in one embodiment about 10 to about 500 cm, and in one embodiment about 50 to about 300 cm.

The catalyst may be supported on a porous support structure such as a foam, felt, wad or a combination thereof. The term “foam” is used herein to refer to a structure with continuous walls defining pores throughout the structure. The term “felt” is used herein to refer to a structure of fibers with interstitial spaces therebetween. The term “wad” is used herein to refer to a structure of tangled strands, like steel wool. The catalyst may be supported on a honeycomb structure.

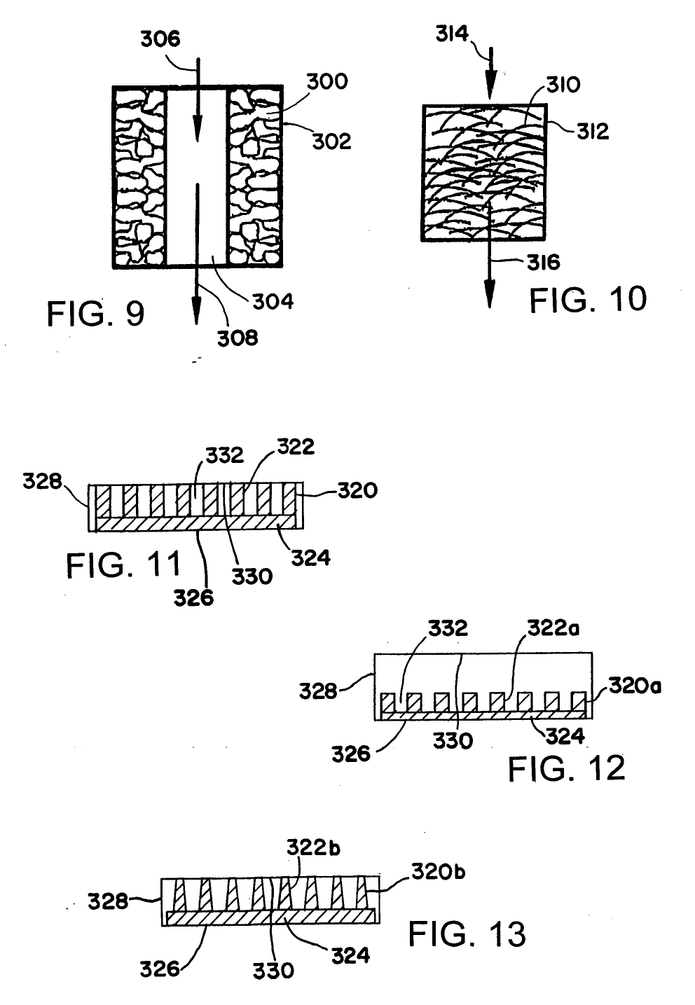

The catalyst may be supported on a flow-by support structure such as a felt with an adjacent gap, a foam with an adjacent gap, a fin structure with gaps, a washcoat on any inserted substrate, or a gauze that is parallel to the flow direction with a corresponding gap for flow. An example of a flow-by structure is illustrated in FIG. 9. In FIG. 9, the catalyst 300 is contained within process microchannel 302. An open passage way 304 permits the flow of fluid through the process microchannel 302 in contact with the catalyst 300 as indicated by arrows 306 and 308.

The catalyst may be supported on a flow-through support structure such as a foam, wad, pellet, powder, or gauze. An example of a flow-through structure is illustrated in FIG. 10. In FIG. 10, the flow-through catalyst 310 is contained within process microchannel 312 and the fluid flows through the catalyst 310 as indicated by arrows 314 and 316.

The support structure for a flow-through catalyst may be formed from a material comprising silica gel, foamed copper, sintered stainless steel fiber, steel wool, alumina, poly(methyl methacrylate), polysulfonate, poly(tetrafluoroethylene), iron, nickel sponge, nylon, polyvinylidene difluoride, polypropylene, polyethylene, polyethylene ethylketone, polyvinyl alcohol, polyvinyl acetate, polyacrylate, polymethylmethacrylate, polystyrene, polyphenylene sulfide, polysulfone, polybutylene, or a combination of two or more thereof. In one embodiment, the support structure may be made of a heat conducting material, such as a metal, to enhance the transfer of heat away from the catalyst.

The catalyst may be directly washcoated on the interior walls of the process microchannels, grown on the walls from solution, or coated in situ on a fin structure. The catalyst may be in the form of a single piece of porous contiguous material, or many pieces in physical contact. In one embodiment, the catalyst may be comprised of a contiguous material and has a contiguous porosity such that molecules can diffuse through the catalyst. In this embodiment, the fluids flow through the catalyst rather than around it. In one embodiment, the cross-sectional area of the catalyst occupies about 1 to about 99%, and in one embodiment about 10 to about 95% of the cross-sectional area of the process microchannels. The catalyst may have a surface area, as measured by BET, of greater than about 0.5 m²/g, and in one embodiment greater than about 2 m²/g.

The catalyst may comprise a porous support, an interfacial layer on the porous support, and a catalyst material on the interfacial layer. The interfacial layer may be solution deposited on the support or it may be deposited by chemical vapor deposition or physical vapor deposition. In one embodiment the catalyst has a porous support, a buffer layer, an interfacial layer, and a catalyst material. Any of the foregoing layers may be continuous or discontinuous as in the form of spots or dots, or in the form of a layer with gaps or holes.

The porous support may have a porosity of at least about 5% as measured by mercury porosimetry and an average pore size (sum of pore diameters divided by number of pores) of about 1 to about 1000 μm. The porous support may be a porous ceramic or a metal foam. Other porous supports that may be used include carbides, nitrides, and composite materials. The porous support may have a porosity of about 30% to about 99%, and in one embodiment about 60% to about 98%. The porous support may be in the form of a foam, felt, wad, or a combination thereof. The open cells of the metal foam may range from about 20 pores per inch (ppi) to about 3000 ppi, and in one embodiment about 20 to about 1000 ppi, and in one embodiment about 40 to about 120 ppi. The term “ppi” refers to the largest number of pores per inch (in isotropic materials the direction of the measurement is irrelevant; however, in anisotropic materials, the measurement is done in the direction that maximizes pore number).

The buffer layer, when present, may have a different composition and/or density than both the porous support and the interfacial layers, and in one embodiment has a coefficient of thermal expansion that is intermediate the thermal expansion coefficients of the porous support and the interfacial layer. The buffer layer may be a metal oxide or metal carbide. The buffer layer may be comprised of Al₂O₃, TiO₂, SiO₂, ZrO₂, or combination thereof. The Al₂O₃ may be a-Al₂O₃, y-Al₂O₃ or a combination thereof. a-Al₂O₃ provides the advantage of excellent resistance to oxygen diffusion. The buffer layer may be formed of two or more compositionally different sublayers. For example, when the porous support is metal, for example a stainless steel foam, a buffer layer formed of two compositionally different sub-layers may be used. The first sublayer (in contact with the porous support) may be TiO₂. The second sublayer may be a-Al₂O₃ which is placed upon the TiO₂. In one embodiment, the a-Al₂O₃ sublayer is a dense layer that provides protection of the underlying metal surface. A less dense, high surface area interfacial layer such as alumina may then be deposited as support for a catalytically active layer.

The porous support may have a thermal coefficient of expansion different from that of the interfacial layer. In such a case a buffer layer may be needed to transition between the two coefficients of thermal expansion. The thermal expansion coefficient of the buffer layer can be tailored by controlling its composition to obtain an expansion coefficient that is compatible with the expansion coefficients of the porous support and interfacial layers. The buffer layer should be free of openings and pin holes to provide superior protection of the underlying support. The buffer layer may be nonporous. The buffer layer may have a thickness that is less than one half of the average pore size of the porous support. The buffer layer may have a thickness of about 0.05 to about 10 μm, and in one embodiment about 0.05 to about 5 μm.

In one embodiment of the invention, adequate adhesion and chemical stability may be obtained without a buffer layer. In this embodiment the buffer layer may be omitted.

The interfacial layer may comprise nitrides, carbides, sulfides, halides, metal oxides, carbon, or a combination thereof. The interfacial layer provides high surface area and/or provides a desirable catalyst-support interaction for supported catalysts. The interfacial layer may be comprised of any material that is conventionally used as a catalyst support. The interfacial layer may be comprised of a metal oxide. Examples of metal oxides that may be used include y-Al₂O₃, SiO₂, ZrO₂, TiO₂, tungsten oxide, magnesium oxide, vanadium oxide, chromium oxide, manganese oxide, iron oxide, nickel oxide, cobalt oxide, copper oxide, zinc oxide, molybdenum oxide, tin oxide, calcium oxide, aluminum oxide, lanthanum series oxide(s), zeolite(s) and combinations thereof. The interfacial layer may serve as a catalytically active layer without any further catalytically active material deposited thereon. Usually, however, the interfacial layer is used in combination with a catalytically active layer. The interfacial layer may also be formed of two or more compositionally different sublayers. The interfacial layer may have a thickness that is less than one half of the average pore size of the porous support. The interfacial layer thickness may range from about 0.5 to about 100 μm, and in one embodiment from about 1 to about 50 μm. The interfacial layer may be either crystalline or amorphous. The interfacial layer may have a BET surface area of at least about 1 m²/g.

The catalyst may be deposited on the interfacial layer. Alternatively, the catalyst material may be simultaneously deposited with the interfacial layer. The catalyst layer may be intimately dispersed on the interfacial layer. That the catalyst layer is “dispersed on” or “deposited on” the interfacial layer includes the conventional understanding that microscopic catalyst particles are dispersed: on the support layer (i.e., interfacial layer) surface, in crevices in the support layer, and in open pores in the support layer.

The catalyst may be supported on an assembly of one or more fins positioned within the process microchannels. Examples are illustrated in FIGS. 11-13. Referring to FIG. 11, fin assembly 320 includes fins 322 which are mounted on fin support 324 which overlies base wall 326 of process microchannel 328. The fins 322 project from the fin support 324 into the interior of the process microchannel 328. The fins 322 extend to and may contact the interior surface of upper wall 330 of process microchannel 328. Fin channels 332 between the fins 322 provide passage ways for fluid to flow through the process microchannel 328 parallel to its length. Each of the fins 322 has an exterior surface on each of its sides, this exterior surface provides a support base for the catalyst. With the inventive process, the reactant composition flows through the fin channels 332, contacts the catalyst supported on the exterior surface of the fins 322, and reacts to form the product. The fin assembly 320a illustrated in FIG. 12 is similar to the fin assembly 320 illustrated in FIG. 11 except that the fins 322a do not extend all the way to the interior surface of the upper wall 330 of the microchannel 328. The fin assembly 320b illustrated in FIG. 13 is similar to the fin assembly 320 illustrated in FIG. 11 except that the fins 322b in the fin assembly 320b have cross sectional shapes in the form of trapezoids. Each of the fins may have a height ranging from about 0.02 mm up to the height of the process microchannel 328, and in one embodiment from about 0.02 to about 10 mm, and in one embodiment from about 0.02 to about 5 mm, and in one embodiment from about 0.02 to about 2 mm. The width of each fin may range from about 0.02 to about 5 mm, and in one embodiment from about 0.02 to about 2 mm and in one embodiment about 0.02 to about 1 mm. The length of each fin may be of any length up to the length of the process microchannel 328, and in one embodiment up to about 10 m, and in one embodiment about 0.5 to about 10 m, and in one embodiment about 0.5 to about 6 m, and in one embodiment about 0.5 to about 3 m. The gap between each of the fins may be of any value and may range from about 0.02 to about 5 mm, and in one embodiment from about 0.02 to about 2 mm, and in one embodiment from about 0.02 to about 1 mm. The number of fins in the process microchannel 328 may range from about 1 to about 50 fins per centimeter of width of the process microchannel 328, and in one embodiment from about 1 to about 30 fins per centimeter, and in one embodiment from about 1 to about 10 fins per centimeter, and in one embodiment from about 1 to about 5 fins per centimeter, and in one embodiment from about 1 to about 3 fins per centimeter. Each of the fins may have a cross-section in the form of a rectangle or square as illustrated in FIGS. 11 or 12, or a trapezoid as illustrated in FIG. 13. When viewed along its length, each fin may be straight, tapered or have a serpentine configuration. The fin assembly may be made of any material that provides sufficient strength, dimensional stability and heat transfer characteristics to permit operation for which the process microchannel is intended. These materials include: steel (e.g., stainless steel, carbon steel, and the like); monel; inconel; aluminum; titanium; nickel; platinum; rhodium; copper; chromium; brass; alloys of any of the foregoing metals; polymers (e.g., thermoset resins); ceramics; glass; composites comprising one or more polymers (e.g., thermoset resins) and fiberglass; quartz; silicon; or a combination of two or more thereof. The fin assembly may be made of an Al₂O₃ forming material such as an alloy comprising Fe, Cr, Al and Y, or a Cr₂O₃ forming material such as an alloy of Ni, Cr and Fe.

In one embodiment, the catalyst may be regenerated. This may be done by flowing a regenerating fluid through the process microchannels in contact with the catalyst. The regenerating fluid may comprise hydrogen or a diluted hydrogen stream. The diluent may comprise nitrogen, argon, helium, methane, carbon dioxide, steam, or a mixture of two or more thereof. The regenerating fluid may flow from the header 104 through the process microchannels and to the footer 106, or in the opposite direction from the footer 106 through the process microchannels to the header 104. The temperature of the regenerating fluid may be from about 50 to about 400° C., and in one embodiment about 200 to about 350° C. The pressure within the process microchannels during this regeneration step may range from about 1 to about 40 atmospheres, and in one embodiment about 1 to about 20 atmospheres, and in one embodiment about 1 to about 5 atmospheres. The residence time for the regenerating fluid in the process microchannels may range from about 0.01 to about 1000 seconds, and in one embodiment about 0.1 second to about 100 seconds.

The catalyst may be regenerated by increasing the molar ratio of H₂ to CO in the reactant composition to at least about 4:1, and in one embodiment at least about 20:1, and flowing the resulting adjusted feed composition through the process microchannels in contact with the catalyst at a temperature in the range from about 120° C. to about 500° C., and in one embodiment in the range from about 180° C. to about 240° C., for a period of time in the range from about 0.5 to about 48 hours, and in one embodiment in the range from about 2 to about 12 hours, to provide the regenerated catalyst.

In one embodiment, the feed composition may be adjusted by interrupting the flow of all feed gases except hydrogen and flowing the hydrogen through the process microchannels in contact with the catalyst. The flow of H₂ may be increased to provide for the same contact time used for the reactant composition comprising H₂ and CO. In one embodiment, the adjusted feed composition may comprise H₂ and is characterized by the absence of CO. Once the catalyst is regenerated, the Fischer-Tropsch process may be continued by contacting the regenerated catalyst with the original reactant composition comprising H₂ and CO.

In one embodiment, the process microchannels may be characterized by having a bulk flow path. The term “bulk flow path” refers to an open path (contiguous bulk flow region) within the process microchannels. A contiguous bulk flow region allows rapid fluid flow through the microchannels without large pressure drops. In one embodiment, the flow of fluid in the bulk flow region is laminar. Bulk flow regions within each process microchannel may have a cross-sectional area of about 0.05 to about 10,000 mm², and in one embodiment about 0.05 to about 5000 mm², and in one embodiment about 0.1 to about 2500 mm². The bulk flow regions may comprise from about 5% to about 95%, and in one embodiment about 30% to about 50% of the cross-section of the process microchannels.

The contact time of the reactants with the catalyst within the process microchannels may range up to about 2000 milliseconds (ms), and in one embodiment from about 10 ms to about 1000 ms, and in one embodiment about 20 ms to about 500 ms. In one embodiment, the contact time may range up to about 300 ms, and in one embodiment from about 20 to about 300 ms, and in one embodiment from about 50 to about 150 ms, and in one embodiment about 75 to about 125 ms, and in one embodiment about 100 ms.

The space velocity (or gas hourly space velocity (GHSV)) for the flow of the reactant composition and product through the process microchannels may be at least about 1000 hr⁻¹ (normal liters of feed/hour/liter of volume within the process microchannels) or at least about 800 ml feed/(g catalyst) (hr). The space velocity may range from about 1000 to about 1,000,000 hr⁻¹, or from about 800 to about 800,000 ml feed/(g catalyst) (hr). In one embodiment, the space velocity may range from about 10,000 to about 100,000 hr⁻¹, or about 8,000 to about 80,000 ml feed/(g catalyst) (hr).

The temperature of the reactant composition entering the process microchannels may range from about 150° C. to about 300° C., and in one embodiment about 180° C. to about 260° C., and in one embodiment about 180° C. to about 230° C.

The temperature of the reactant composition and product within the process microchannels may range from about 200° C. to about 300° C., and in one embodiment from about 220° C. to about 270° C., and in one embodiment from about 220° C. to about 250° C.

The temperature of the product exiting the process microchannels may range from about 200° C. to about 300° C., and in one embodiment about 220° C. to about 270° C., and in one embodiment about 220° C. to about 250° C.

The pressure within the process microchannels may be at least about 5 atmospheres, and in one embodiment at least about 10 atmospheres, and in one embodiment at least about 15 atmospheres, and in one embodiment at least about 20 atmospheres, and in one embodiment at least about 25 atmospheres, and in one embodiment at least about 30 atmospheres. In one embodiment the pressure may range from about 5 to about 75 atmospheres, and in one embodiment from about 10 to about 50 atmospheres, and in one embodiment from about 10 to about 30 atmospheres, and in one embodiment from about 10 to about 25 atmospheres, and in one embodiment from about 15 to about 25 atmospheres.

The pressure drop of the reactants and/or products as they flow through the process microchannels may range up to about 15 atmospheres per meter of length of the process microchannel (atm/m), and in one embodiment up to about 10 atm/m, and in one embodiment up to about 5 atm/m.

The reactant composition entering the process microchannels is typically in the form of a vapor, while the product exiting the process microchannels may be in the form of a vapor, a liquid, or a mixture of vapor and liquid. The Reynolds Number for the flow of vapor through the process microchannels may be in the range of about 10 to about 4000, and in one embodiment about 100 to about 2000. The Reynolds Number for the flow of liquid through the process microchannels may be about 10 to about 4000, and in one embodiment about 100 to about 2000.

The heat exchange fluid entering the heat exchange channels may be at a temperature of about 150° C. to about 300° C., and in one embodiment about 150° C. to about 270° C. The heat exchange fluid exiting the heat exchange channels may be at a temperature in the range of about 220° C. to about 270° C., and in one embodiment about 230° C. to about 250° C. The residence time of the heat exchange fluid in the heat exchange channels may range from about 50 to about 5000 ms, and in one embodiment about 100 to about 1000 ms. The pressure drop for the heat exchange fluid as it flows through the heat exchange channels may range up to about 10 atm/m, and in one embodiment from about 0.01 to about 10 atm/m, and in one embodiment from about 0.02 to about 5 atm/m. The heat exchange fluid may be in the form of a vapor, a liquid, or a mixture of vapor and liquid. The Reynolds Number for the flow of vapor through the heat exchange channels may be from about 10 to about 4000, and in one embodiment about 100 to about 2000. The Reynolds Number for the flow of liquid through heat exchange channels may be from about 10 to about 4000, and in one embodiment about 100 to about 2000.

The conversion of CO may be about 40% or higher per cycle, and in one embodiment about 50% or higher, and in one embodiment about 55% or higher, and in one embodiment about 60% or higher, and in one embodiment about 65% or higher, and in one embodiment about 70% or higher. The term “cycle” is used herein to refer to a single pass of the reactants through the process microchannels.

The selectivity to methane in the product may be about 25% or less, and in one embodiment about 20% or less, and in one embodiment about 15% or less, and in one embodiment about 12% or less, and in one embodiment about 10% or less.

The yield of product may be about 25% or higher per cycle, and in one embodiment about 30% or higher, and in one embodiment about 40% or higher per cycle.

In one embodiment, the conversion of CO is at least about 50%, the selectivity to methane is about 15% or less, and the yield of product is at least about 35% per cycle.

The product formed by the inventive process may comprise a gaseous product fraction and a liquid product fraction. The gaseous product fraction may include hydrocarbons boiling below about 350° C. at atmospheric pressure (e.g., tail gases through middle distillates). The liquid product fraction (the condensate fraction) may include hydrocarbons boiling above about 350° C. (e.g., vacuum gas oil through heavy paraffins).

The product fraction boiling below about 350° C. may be separated into a tail gas fraction and a condensate fraction, e.g., normal paraffins of about 5 to about 20 carbon atoms and higher boiling hydrocarbons, using, for example, a high pressure and/or lower temperature vapor-liquid separator, or low pressure separators or a combination of separators. The fraction boiling above about 350° C. (the condensate fraction) may be separated into a wax fraction boiling in the range of about 350° C. to about 650° C. after removing one or more fractions boiling above about 650° C. The wax fraction may contain linear paraffins of about 20 to about 50 carbon atoms with relatively small amounts of higher boiling branched paraffins. The separation may be effected using fractional distillation.

The product formed by the inventive process may include methane, wax and other heavy high molecular weight products. The product may include olefins such as ethylene, normal and iso-paraffins, and combinations thereof. These may include hydrocarbons in the distillate fuel ranges, including the jet or diesel fuel ranges.

Branching may be advantageous in a number of end-uses, particularly when increased octane values and/or decreased pour points are desired. The degree of isomerization may be greater than about 1 mole of isoparaffin per mole of n-paraffin, and in one embodiment about 3 moles of isoparaffin per mole of n-paraffin. When used in a diesel fuel composition, the product may comprise a hydrocarbon mixture having a cetane number of at least about 60.

Higher molecular weight products, for example waxes, may either be isolated and used directly, or reacted to form lower molecular weight products. For example, high molecular weight products may be hydrocracked to provide lower molecular weight products, increasing the yield of liquid combustible fuels. Hydrocracking refers to a catalytic process, usually carried out in the presence of free hydrogen, in which the cracking of the larger hydrocarbon molecules is a primary purpose of the operation. Catalysts used in carrying out hydrocracking operations are well known in the art; see, for example, U.S. Pat. Nos. 4,347,121 and 4,810,357, which are incorporated herein by reference, for their descriptions of hydrotreating, hydrocracking, and catalysts used in each process. The product formed by the inventive process may be further processed to form a lubricating base oil or diesel fuel. For example, the product made by the inventive process may be hydrocracked and then subjected to distillation and/or catalytic isomerization to provide a lubricating base oil, diesel fuel, and the like.

The hydrocarbon products made by the inventive process may be hydroisomerized using the process disclosed in U.S. Pat. Nos. 6,103,099 or 6,180,575; hydrocracked and hydroisomerized using the process disclosed in U.S. Pat. Nos. 4,943,672 or 6,096,940; dewaxed using the process disclosed in U.S. Pat. No. 5,882,505; or hydroisomerized and dewaxed using the process disclosed in U.S. Pat. Nos. 6,013,171, 6,080,301 or 6,165,949. These patents are incorporated herein by reference for their disclosures of processes for treating Fischer-Tropsch synthesized hydrocarbons and the resulting products made from such processes.

### EXAMPLE 1

A Fischer-Tropsch reaction is conducted in a process microchannel reactor using a Co/Re—Al₂O₃ catalyst (Catalyst A) with a Co to Re molar ratio of 21:1. The process microchannel is fabricated from SS316 stainless steel. The process microchannel has a height or gap of 0.51 mm, width of 1.27 cm and length of 8 cm. The process microchannel has a reaction zone containing the catalyst, the catalyst being in the form of a bed of particulate solids. The reaction zone has a length of 2.5 to 3 cm. The remainder of the process microchannel is either open or filled with quartz wool wadding material to retain the catalyst. The wadding material takes up 1 cm on either side of the catalyst bed. Temperature in the process microchannel and the catalyst bed is determined via a series of thermocouples placed in wells that are located 0.75 cm, 2.0 cm, 3.3 cm and 4.6 cm upstream from the outlet of the process microchannel. The process microchannel has an open space of 2 to 3 cm upstream from the catalyst bed to permit preheating the feed stream material. The process microchannel is externally jacketed so that heat exchange fluid (e.g., water or a heat transfer oil) can be circulated cocurrently or counter currently relative to the flow of fluid in the process microchannel.

The process microchannel is installed in a test enclosure. The gas mixtures required for catalyst activation and reactor operation are provided via a bank of mass flow controllers. The product stream from the reactor is a two phase stream which is directed to a cold trap where a condensable fraction is collected. Once the condensable fraction is removed from the product stream, the remaining product gas passes through a back pressure regulator to an on-line gas chromatograph. The test set-up includes a feed by-pass which allows for checking the feed gas composition periodically. The conversion of carbon monoxide is determined using the inlet flow of carbon monoxide, the fraction reported in the outlet dry gas, and the outlet dry gas flow rate. Selectivity to methane is calculated by dividing the outlet flow of methane by the amount of carbon monoxide converted.

The catalyst is prepared by first calcining a sample of Al₂O₃ support at 650° C. and then performing 3 impregnations using an aqueous solution containing 30% by weight Co (from cobalt nitrate) and 4.5% by weight Re (from perrhenic acid). After the first and second impregnations the catalyst is dried in an oven for 12 hours at 90° C. After the final impregnation the catalyst is calcined by heating the catalyst to 350° C. at a rate of 1° C. per minute and then holding at 350° C. for 3 hours. The catalyst has a particle size in the range of 177 to 250 microns. The metal dispersion is 5.4%.

A 0.19 gram sample of the catalyst is loaded into the process microchannel forming a 3 cm deep bed of catalyst. The catalyst is activated by alternating reduction (3 cycles) and oxidation (2 cycles). This may be referred to as redox cycling. In the first reduction cycle 41 standard cubic centimeters (sccm) of 5 vol % hydrogen gas (balance He) flow over the catalyst. The pressure within the process microchannel is atmosphere pressure. The process microchannel is heated from 20° C. to 250° C. over a period of 3.8 hours, and then held at that temperature for an additional 8 hours. The temperature is increased to 400° C. over a period of 2.5 hours and then held at that temperature for additional 12 hours. The 5 vol % hydrogen gas flow is then replaced with He only at the same flow rate and the process microchannel is allowed to cool to ambient temperature over a period of 13.2 hours. This reduction cycle is followed by an oxidation cycle in which 30 sccm of He gas containing 2 vol % oxygen flows over the catalyst. The temperature is increased from 22° C. to 350° C. at a rate of 1.5° C. per minute. The process microchannel is then held at 350° C. for 2 hours. The 2 vol % oxygen stream is then replaced with He only at the same flow rate and the process microchannel is allowed to cool to ambient temperature. The total time for this cooling and purging step is 18.1 hours.

The first oxidation step is then followed by a second reduction cycle that is the same in all respects to the first reduction cycle with the exception that the final cooling and purging step lasts 24 hours. The second reduction cycle is followed by a second oxidation cycle. This cycle is the same in all respects to the first oxidation cycle with the exception that the cooling and purging step lasts 22 hours. The last step in catalyst activation involves a third reduction cycle which includes flowing 41 sccm of the 5 vol % hydrogen gas through the process microchannel. The process microchannel is heated from 24° C. to 250° C. at a rate of 1° C. per minute. The process microchannel temperature is held at 250° C. for one half hour. The process microchannel is then heated to 375° C. at a rate of 1° C. per minute and held at this temperature for 12 hours. The process microchannel is cooled to 160° C. and pressurized to 400 pounds per square inch gage (psig) (28.2 atm absolute) under a 5 vol % hydrogen gas (balance He) over a period of about 4 hours. In this manner the catalyst is activated and the process microchannel is ready for operation.

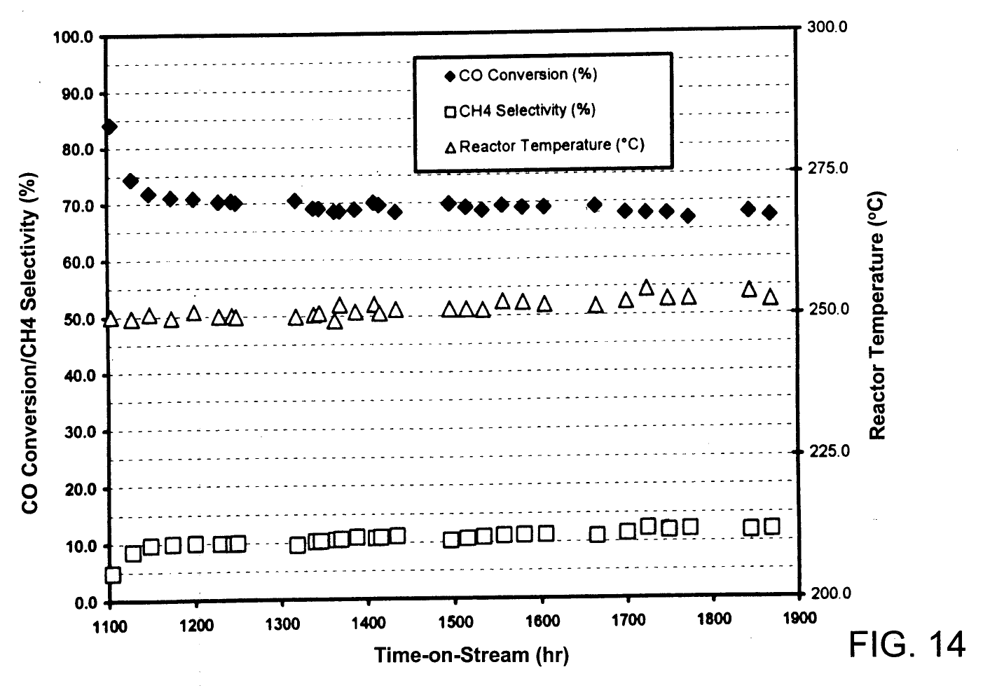

The feed composition for the Fischer-Tropsch reaction process is a gaseous mixture containing hydrogen (61 vol %), carbon monoxide (35 vol %) and helium (4 vol %). The process microchannel is brought on line at an internal pressure of 21 atm and a temperature of 180° C. The process microchannel temperature and the contact time are adjusted until a steady state condition is reached at a temperature of of 225° C., and a contact time of 288 millisecond (ms) (12,500 hr⁻¹ GHSV). At this point the catalyst has 320 hours time-on-stream and the conversion of carbon monoxide is 47% and the selectivity to methane 10%. For the next 481 hours time-on-stream the pressure and feed flow rate are fixed. At 801 hours time-on-stream the pressure is increased from 21 atm to 41 atm. The increase in pressure increases the conversion of carbon monoxide to 56% and reduces the methane selectivity to 7%. At 933 hours time-on-stream a temperature ramp is used to take the process microchannel from 225° C. to 250° C. The contact time is increased from 288 ms to 308 ms (11,688 hr⁻¹ GHSV). The process microchannel reaches thermal steady state at 1104 hours time-on-stream. The process microchannel is operated under constant conditions for the next 765 hours over which time the average values of CO conversion and methane selectivity are 69.7% and 10.5%, respectively. This is shown in FIG. 14.

### EXAMPLE 2

A Fischer-Tropsch reaction is conducted in a process microchannel using a powdered Co/Re—Al₂O₃ catalyst (Catalyst B) with a Co to Re molar ratio of 21:1. The catalyst has a loading of cobalt of 30% by weight and a loading of rhenium of 4.5% by weight. The process microchannel has a height or gap of 0.686 mm, width of 1.27 cm and length of 8.1 cm. The catalyst bed is 2.18 cm deep. Temperature in the catalyst bed is estimated via a series of five thermocouples located on the skin of the process microchannel. The process microchannel is externally jacketed so that heat exchange fluid (e.g., water or a heat transfer oil) can be circulated cocurrently or counter currently relative to the flow of fluid in the process microchannel.

The catalyst B is prepared using a particulate acidic gamma alumina support having a 50 to 80 mesh size. Prior to preparation of the catalyst, the support has a pore volume is 0.8 ml/g. After preparation the particle size and distribution of the catalyst is assessed using a laser scattering technique. The median particle size is 275 microns. The alumina support is impregnated four times with a solution containing cobalt nitrate and perrhnic acid to provide a loading of 30% by weight and a Re loading of 45% by weight. After each impregnation the catalyst is dried in an oven at 90° C. for 14 hours and then calcined by heating at a rate of 5° C. per minute to 300° C. and holding the temperature at 300° C. for 3 hours.

Two samples of Catalyst B are prepared. These are identified as samples B-1 and B-2. Catalyst sample B-1 is loaded in the process microchannel to provide a catalyst bed. The catalyst is reduced by first allowing 50 sccm of pure hydrogen to flow through the process microchannel in contact with the catalyst and then raising the temperature in the catalyst bed to 250° C. at a rate of 1° C. per minute. The catalyst is held at 250° C. for 30 minutes and then ramped to 400° C. at a rate of 0.5° C. per minute. The catalyst is held at 400° C. for 12 hrs. This process may be referred to as a direct reduction process.

Catalyst sample B-2 is reduced under 50 sccm of a gas containing 10% hydrogen (remainder helium). The catalyst is heated to 250° C. at a rate of 1° C./min and held at this temperature for 6 hours. The bed is then heated to 400° C. at a rate of 1° C./min and held at 400° C. for 12 hours. The reactor is then allowed to cool down to 50° C. under a flow of helium. A flow of 30 sccm of 2% oxygen (remainder helium) is passed over the catalyst and the bed it heated to 350° C. at a rate of 2° C. per minute, and then held at 350° C. for 2 hours. The reactor is allowed to cool to 50° C. under a flow of helium. The foregoing steps are repeated. The catalyst is then reduced under 50 sccm of a gas containing 10% hydrogen (remainder helium) by increasing the temperature to 375° C. at a rate of 1° C./min and then holding the temperature at 375° C. for 12 hours. The reactor is allowed to cool to 160° C. under flowing helium.

Samples B-1 and B-2 of Catalyst B are characterized via temperature programmed reduction (TPR). The mass of catalyst used for each TPR run is between 50 and 70 milligrams (mg) and the particle size is between 50 and 80 mesh. The catalyst samples are first degassed in-situ under argon at 150° C. for 30 min then cooled to 50° C. The catalyst samples are then subjected to two reduction and oxidation cycles (redox) prior to TPR. Each reduction step uses the following procedure:
1) Purge the process microchannel with Ar (50 cc/min) for 5 min at room temperature.
2) Switch gas to H₂ (30 cc/min).
3) Ramp temperature to 400° C. (4° C./min).
4) Hold temperature for 3 hour at 400° C.
5) Cool the process microchannel under H₂ (30 cc/min) to room temperature.
6) Purge the process microchannel with Ar for 30 min at room temperature.

Each oxidation step uses the following procedure:
1) Switch gas to 10% by volume O₂ (remainder He) and flow the gas through the process microchannel at a rate of 30 cc/min.
2) Ramp temperature to 350° C. (4° C./min).
3) Hold temperature for 2 hour at 350° C.
4) Cool the process microchannel to room temperature by flowing 10% by volume O₂ (remainder He) through the process microchannel.
5) Purge the process microchannel with Ar (50 cc/min) for 30 min at room temperature.

The TPR procedure uses the following steps:
1) Switch gas to 10% by volume H₂ (remainder Ar) and flow gas through process microchannel at a rate of 30 cc/min for 15 min at 50° C.
2) Ramp temperature to 600° C. (10° C./min).
3) Hold temperature for 1 h at 600° C.
4) Cool to room temperature under Ar.

The reactor described above is installed in a test enclosure and the gas mixtures required for catalyst activation and reactor operation are provided via premixed gasses. A two phase product stream flows from the reactor and is directed to a cold trap in wherein a condensable fraction is collected. Once the condensable fraction is removed from the product stream the remaining product gases pass through a back pressure regulator to an on-line gas chromatograph. The conversion of carbon monoxide is determined using the inlet flow of carbon monoxide, the fraction reported in the outlet dry gas, and the outlet dry gas flow rate. Selectivity to methane is calculated by dividing the outlet flow of methane by the amount of carbon monoxide converted. The feed composition is a gaseous mixture of hydrogen (64 vol %), carbon monoxide (32 vol %) and argon (4 vol %). Contact time is 270 ms and the reactor pressure is 26.9 atm. Reactive testing is conducted at 210° C. and 220° C.

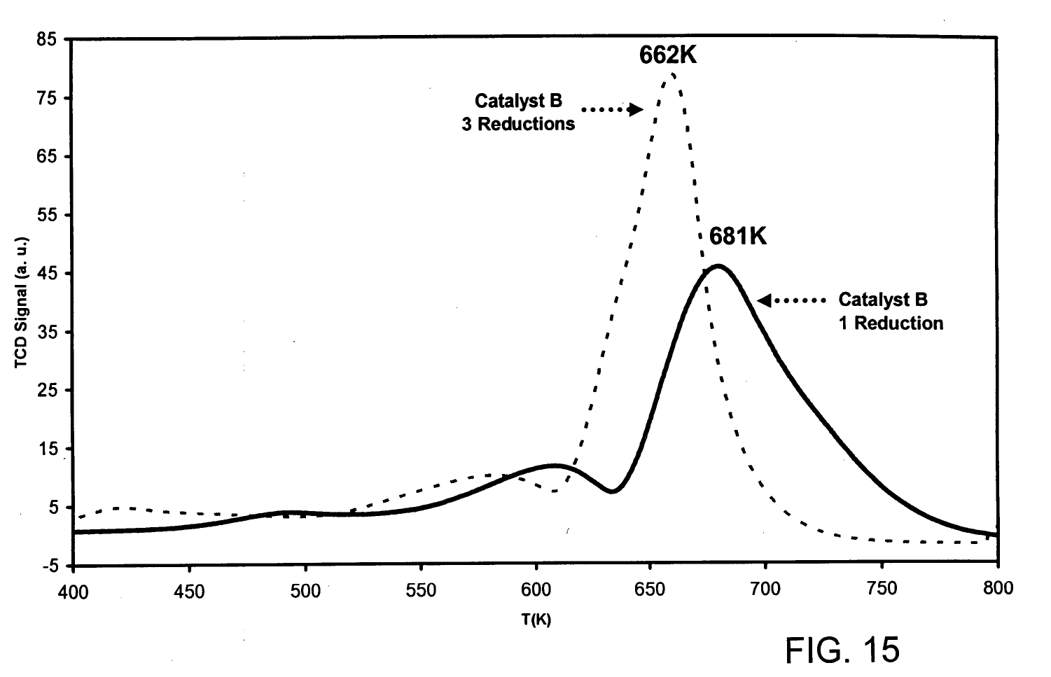

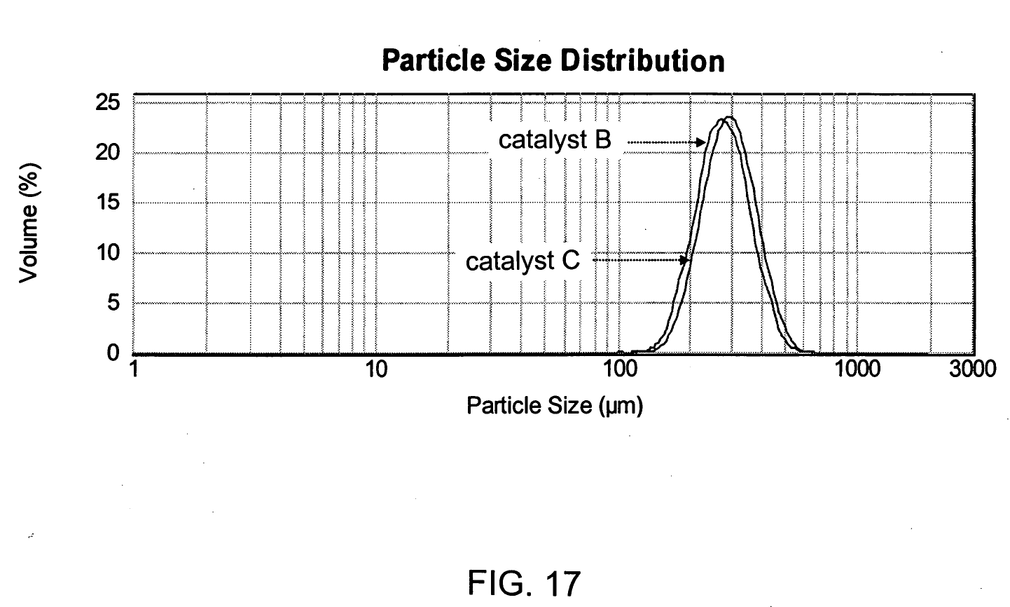

The particle size distribution for Catalyst B is shown in FIG. 17. The importance of the number of redox cycles is indicated by reactive testing results for catalyst sample B-1 and catalyst sample B-2. Catalyst samples B-1 and B-2 are taken from the same batch, but sample B-2 undergoes 3 redox cycles while sample B-1 undergoes 1 redox cycle. Sample B-2 out performs sample B-1 in carbon monoxide conversion and selectivity to methane. The TPR profile (FIG. 15) indicates that the redox procedure facilitates the reduction of Co and Re. While not wishing to be bound by theory, it is believed that the changes induced by the redox treatment are changes in the dispersion and repartition of the noble metals on the surface of the catalyst support.

### EXAMPLE 3

A pair of catalysts, Catalysts C and D, are prepared using an acidic gamma alumina support with a Co to Re molar ratio of 21:1. The loading of cobalt is 30% by weight, and the loading of rhenium is 4.5% by weight.

Catalyst C is prepared using acidic alumina support that has a BET surface area of 260 m²/g and a pore volume of 0.8 cc/g. The alumina is ground in a pestle, and the resulting powder is passed through a sieve assembly containing a 50 mesh, 80 mesh, and a collector. The ground alumina (50/80 mesh) is sieved a second time to the same particle size (50/80 mesh) using an automatic shaker. The sieving duration is 10 min for each 100 grams of alumina.

The recovery rate in the desired size cut is 50% by weight. The ground alumina (50/80 mesh) is placed in a Pyrex dish (500 grams of alumina per dish), calcined in a box furnace by increasing the temperature from room temperature to 350° C. at a rate of 2° C./min, and then held at 350° C. for 4 hours. After calcination, the furnace is cooled to room temperature. Batches of calcined alumina are kept in a bottle and stored in a desiccator. After calcination the moisture content of the alumina is between 1% and 3% by weight.

The catalyst contains 30% by weight Co and 4.5% by weight Re. The catalyst is prepared using 350 grams of the foregoing particulate alumina support, 1055.8 grams of Cobalt (II) nitrate hexahydrate 98+ (Spectrum chemical, C1315-18), and 63 grams of perrhenic acid 99.99% 65-70 wt %. (Aldrich, 261963). Four impregnation cycles are used to impregnate the alumina support with Co and Re. For each impregnation cycle one-fourth of the Co precursor and one-fourth of the Re precursor are used. For each impregnation a new solution is prepared. For the first impregnation the powdered alumina support pore volume is 0.8 cc/gram. The following solution is used for the first impregnation: Cobalt (II) nitrate hexahydrate 98+: 264.0 g (Spectrum chemical; C1315-18); perrhenic acid 99.99% 65-70 wt %: 15.7 g (Aldrich; 261963); sufficient deionized (DI) water to make 245-270 ml of solution. The solution is heated to 70° C. to accelerate the dissolution of cobalt.

The powdered alumina support (350 grams) is placed in a mixing container with two baffles. The container is then placed in an angled rotating mixer and allowed to mix for at least 5 min at a speed of 20-30 rpm. The rotating mixture is occasionally stirred using a spatula. While the powdered alumina is rotating, 1 to 2 grams of solution are added by spraying and allowing the solution to impregnate uniformly. When more than 25% of solution is impregnated, the rotation speed is increased to 40-70 rpm. After all solution is impregnated, the powder is allowed to mix for an additional 30 min. After the first impregnation, the powder sample is placed in a Pyrex dish and calcined by placing the dish in a drying furnace pre-heated to 90° C. and leaving it there for 14 hours. After the initial heating period has elapsed, the furnace is heated to 300° C. at a rate of 5° C./min. The sample is then held at 300° C. for 4 hours. The sample is then cooled to 40° C. at a rate of 5° C. per min.

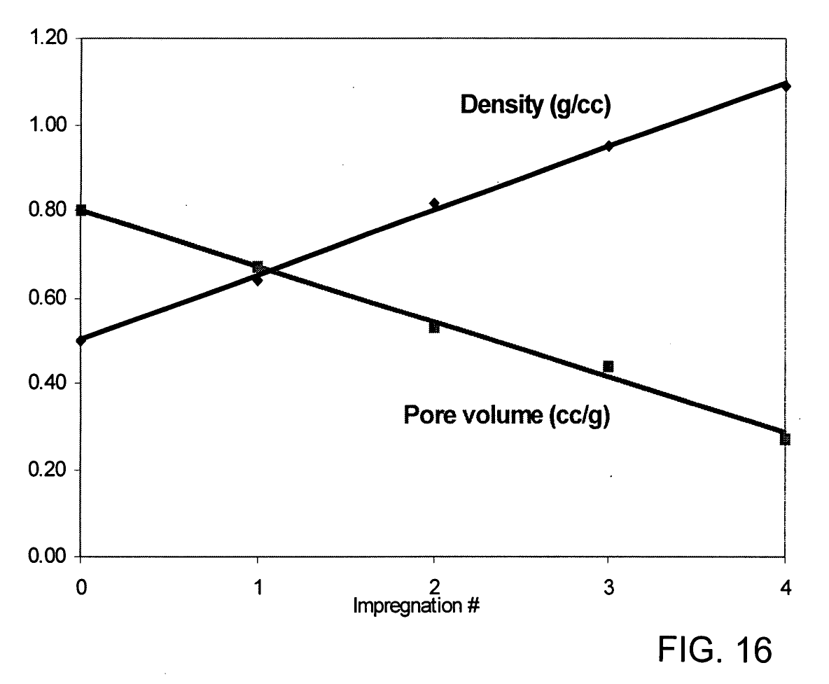

The next three impregnations are performed in the same way as the first impregnation. For each impregnation the same amount of Co and Re are added. The volume of the solution is adjusted to approximately 95% of the pore volume of the catalyst. FIG. 16 shows the relation between density and pore volume after each impregnation step. The particle size distribution for Catalyst C is shown in FIG. 17. After the final impregnation and drying steps, Catalyst C has the following proprieties:
BET area, cm²/g: 100-120
Pore volume, cc/g: 0.25-0.35
Metal dispersion: 5-7% (pulse technique)
Density: 1.05-1.25 g/cm

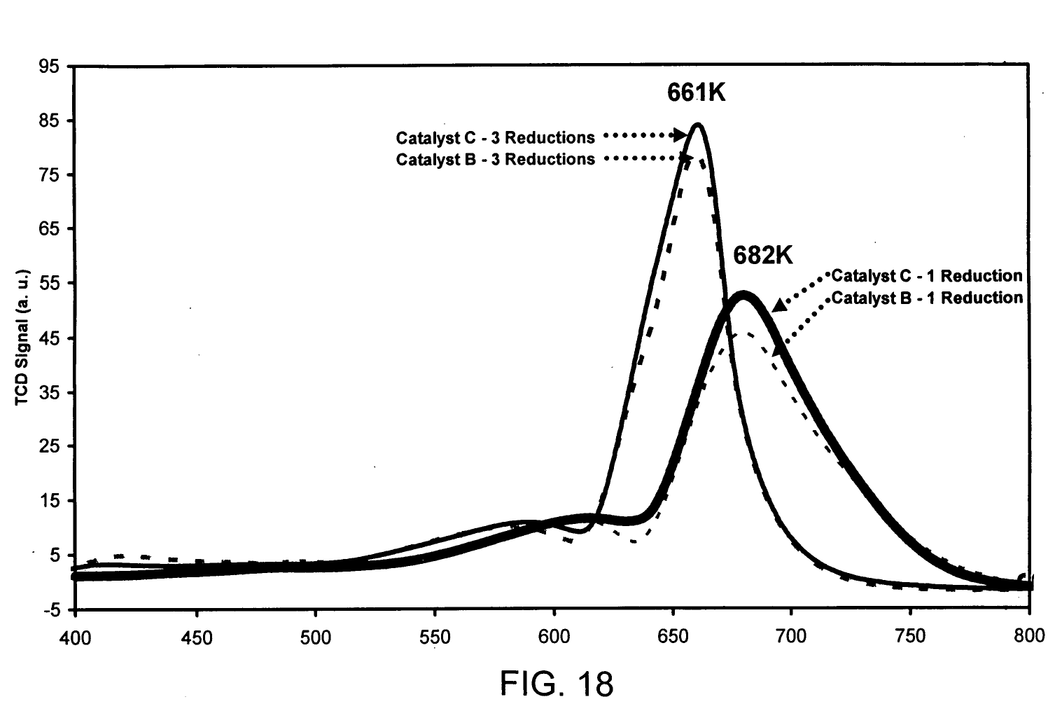

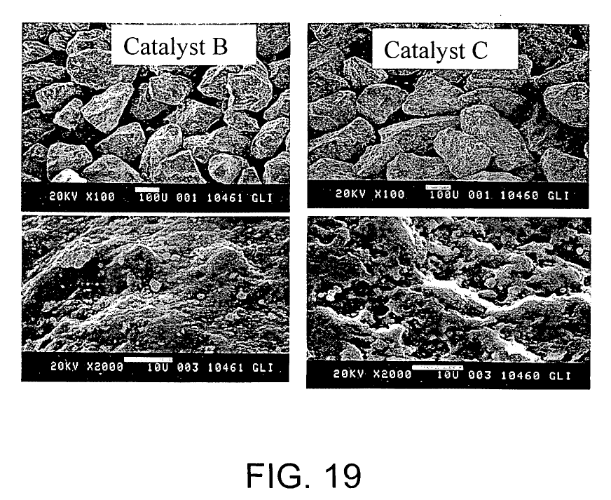

TPR data is gathered for catalyst C in a manner similar to that reported for Example 2. A comparison between a sample of Catalyst C and a sample of Catalyst B is reported in FIG. 18. SEM photographs (FIG. 19) compare the morphology of Catalysts B and C.

Catalyst C in the form of a catalyst bed in a reactor is reduced under 50 sccm of a gas containing 10% hydrogen (remainder helium). The catalyst is heated to 250° C. at a rate of 1° C./min and held at 250° C. for 6 hours. The catalyst bed is then heated to 400° C. at a rate of 1° C./min and held at 400° C. for 12 hours. The reactor is then cooled to 50° C. under a flow of helium. A flow of 30 sccm of 2% oxygen (remainder helium) is passed over the catalyst and the bed is heated to 350° C. at a rate of 2° C./min and held at 350° C. for 2 hours. The reactor is cooled to 50° C. under a flow of helium. The above steps are repeated. The catalyst is then reduced under 50 sccm of a gas containing 10% hydrogen (remainder helium) by increasing the temperature to 375° C. at the rate of 1° C./min and then holding the temperature at 375° C. for 12 hours. The reactor is cooled to 160° C. under flowing helium.

Catalyst D is reduced via the same procedure used for sample B-1. Catalysts C and D are tested using the same process microchannel and procedure described in Example 2. Representative samples of catalysts B, C and D are tested under Fischer-Tropsch conditions and in the process microchannel test set-up described in Example 2. Catalysts B and C exhibit similar performance at 210° C. Catalyst D, prepared from the same starting materials and by the same method as catalyst C but activated via the direct reduction method, shows relatively poor performance. The results are provided in Table 1.

TABLE 1

| | Sample | | | | | | |
|---|---|---|---|---|---|---|---|
| | (—) | C | D | B-2 | C | D | B-2 |
| In-Reactor | (—) | 3 | 1 | 3 | 1 | 1 | 3 |
| Reductions | | | | | | | |
| FT Reaction | (° C.) | 210 | 210 | 210 | 220 | 220 | 220 |
| Temperature | | | | | | | |
| Conversion of Carbon | (%) | 62.3 | 38.1 | 62.2 | 68.9 | 52.2 | 73.5 |
| Monoxide | | | | | | | |
| Selectivity | (%) | 10.8 | 14.5 | 9.3 | 13.6 | 20.0 | 11.4 |
| to Methane | | | | | | | |

### EXAMPLE 4

A Co-Re catalyst prepared in a manner similar to that described in Example 1 is tested in an oil-cooled process microchannel. The process microchannel has a width of 2.54 cm, and a height or gap of 0.762 mm. The catalyst has a 70 to 100 mesh size and forms a bed 4.65 cm deep in the process microchannel. The process is conducted using a feed gas composition containing 10% nitrogen, the remainder being hydrogen and CO. The hydrogen to CO molar ratio is 1.6:1. The process is conducted using a contact time of 257 ms, temperature of 228.6° C., and pressure of 295 to 313 psig (21.1-22.3 atm absolute pressure). After 236 hours of time-on-stream the conversion of CO declines from 66.7% to 54%. Over the same period the selectivity to methane increases from 9.2% to 9.9%. To re-activate the catalyst, the CO and nitrogen flows are terminated and the hydrogen flow is adjusted to maintain the contact time at 257 ms. The process microchannel is heated to 400° C. at a rate of 1° C. per minute and held at 400° C. for 12 hours. The temperature is then reduced to 160° C. over a period of 6 hours. The nitrogen and CO are re-introduced in steps and the extra hydrogen is reduced in steps over a period of 1 hour. The process microchannel is then maintained at 160° C. for 12 hours. The temperature is then increased to 180° C. at a rate of 1° C./15 minutes and held at 180° C. for 24 hours. The process microchannel temperature is then increased from 180° C. to 200° C. at rate of 1° C./15 minutes and held at 200° C. for 24 hours. The process microchannel is then heated at a rate of 1° C./15 minutes for 3 hours then at a rate of 1° C./hour to the operating temperature of 229° C. The conversion of CO is returned to a value of 67.5% and the selectivity to methane is 10.4%.

### EXAMPLE 5

The catalyst described in Example 4 declined in activity over a period of 218 hours time-on-stream from 67.5% conversion of CO to 53%. Keeping the total inlet pressure, reactor temperature and contact time the same the flows of CO and hydrogen are adjusted such that the hydrogen to CO ratio is 9:1. The feed gas contains 10% nitrogen by volume. This condition is maintained for 1.5 hours, then the ratio of hydrogen to CO is readjusted to 1.6:1 over a period of 45 minutes. The conversion of CO returns to 67.9% and the selectivity to methane is 9.9%.

While the invention has been explained in relation to various detailed embodiments, it is to be understood that various modifications thereof will become apparent to those skilled in the art upon reading the specification. Therefore, it is to be understood that the invention disclosed herein is intended to cover such modifications as fall within the scope of the appended claims.

1. A catalyst, comprising:
cobalt and optionally a co-catalyst and/or promoter on a support, the catalyst being made by the process comprising:
(A) contacting the support with the cobalt and optionally the co-catalyst and/or promoter to form a supported catalyst;
(C) oxidizing the supported catalyst; and
(D) reducing the supported catalyst.

2. The catalyst of claim 1, further comprising subsequent to (A) but prior to (C) the following:
(B) reducing the supported catalyst.

3. The catalyst of claim 1, further comprising subsequent to (D) the following:
(E) oxidizing the supported catalyst; and
(F) reducing the supported catalyst.

4. A catalyst, comprising:
cobalt and optionally a co-catalyst and/or promoter on a support, the catalyst being made by the process comprising:
(A) contacting the support with the cobalt and optionally the co-catalyst and/or promoter to form a supported catalyst;
(B) reducing the supported catalyst;
(C) oxidizing the supported catalyst;
(D) reducing the supported catalyst;
(E) oxidizing the supported catalyst; and
(F) reducing the supported catalyst.

5. The catalyst of claim 1 wherein (A) comprises:
(A-1) contacting the support with a composition comprising at least one cobalt compound and optionally at least one co-catalyst and/or promoter; and
(A-2) calcining the contacted support from (A-1).

6. The catalyst of claim 2 wherein (A) further comprises:
(A-3) contacting the support from (A-2) with another composition comprising at least one cobalt compound and optionally at least one co-catalyst and/or promoter; and
(A-4) calcining the contacted support from (A-3).

7. The catalyst of claim 2 wherein (B) comprises:
(B-1) heating the supported catalyst in the presence of a reducing fluid to a first temperature in the range from about 100° C. to about 500° C. over a period of time in the range from about 0.5 to about 12 hours;
(B-2) maintaining the supported catalyst at the first temperature in the presence of the reducing fluid for a period of time in the range from about 2 to about 25 hours;
(B-3) heating the supported catalyst from (B-2) in the presence of the reducing fluid to a second temperature in the range from about 300° C. to about 600° C. over a period of time in the range from about 0.5 to about 12 hours;
(B-4) maintaining the supported catalyst in the presence of the reducing fluid at the second temperature for a period of time in the range from about 0.5 to about 24 hours; and
(B-5) contacting the supported catalyst from (B-4) with an inert fluid and cooling the supported catalyst to ambient temperature over a period of about 1 to about 48 hours.

8. The catalyst of claim 1 wherein (C) comprises:
(C-1) heating the supported catalyst in the presence of an oxidizing fluid to a first temperature in the range from about 150° C. to about 650° C. over a period of time in the range from about 0.5 to about 12 hours;
(C-2) maintaining the supported catalyst at the first temperature in the presence of the oxidizing fluid for a period of time in the range from about 0.5 to about 10 hours; and
(C-3) contacting the supported catalyst with an inert fluid and cooling the supported catalyst to ambient temperature over a period of time in the range from about 1 to about 48 hours.

9. The catalyst of claim 1 wherein (D) comprises:
(D-1) heating the supported catalyst in the presence of a reducing fluid to a first temperature in the range from about 100° C. to about 500° C. over a period of time in the range from about 0.5 to about 10 hours;
(D-2) maintaining the supported catalyst at the first temperature in the presence of the reducing fluid for a period of time in the range from about 2 to about 25 hours;
(D-3) heating the supported catalyst from (D-2) in the presence of the reducing fluid to a second temperature in the range from about 300° C. to about 600° C. over a period of time in the range from about 0.5 to about 12 hours;
(D-4) maintaining the supported catalyst at the second temperature in the presence of the reducing fluid for a period of time in the range from about 0.5 to about 24 hours; and
(D-5) contacting the supported catalyst with an inert fluid and cooling the supported catalyst to ambient temperature over a period of time in the range from about 1 to about 48 hours.

10. The catalyst of claim 3 wherein (E) comprises:
(E-1) heating the supported catalyst in the presence of an oxidizing fluid to a first temperature in the range from about 150° C. to about 650° C. over a period of time in the range from about 0.5 to about 12 hours;
(E-2) maintaining the supported catalyst at the first temperature in the presence of the oxidizing fluid for a period of time in the range from about 0.5 to about 10 hours; and
(E-3) contacting the supported catalyst with an inert fluid and cooling the supported catalyst to ambient temperature over a period of time in the range from about 1 to about 48 hours.

11. The catalyst of claim 3 wherein (F) comprises:
(F-1) heating the supported catalyst in the presence of a reducing fluid to a first temperature in the range from about 100° C. to about 500° C. over a period of time in the range from about 0.5 to about 20 hours;
(F-2) maintaining the supported catalyst at the first temperature in the presence of the reducing fluid for a period of time in the range from about 0.5 to about 48 hours;
(F-3) heating the supported catalyst from (F-2) in the presence of the reducing fluid to a second temperature in the range from about 100° C. to about 600° C. over a period of time in the range from about 0.5 to about 12 hours;
(F-4) maintaining the supported catalyst in the presence of the reducing fluid at the second temperature for a period of time in the range from about 0.5 to about 48 hours; and
(F-5) contacting the supported catalyst with the reducing fluid at a pressure in the range from about 0.7 to about 75 atm and cooling the supported catalyst to a temperature in the range from about 60° C. to about 250° C. over a period of time in the range from about 0.5 to about 40 hours.

12. The catalyst of claim 1 wherein the catalyst comprises cobalt supported on alumina.

13. The catalyst of claim 1 wherein the active portion of the catalyst comprises a composition represented by the formula
CoM¹ₐ M²_{b}Oₓ
wherein: M¹ is Fe, Ni, Ru, Re, Os or a mixture thereof; M² is Li, B, Na, K, Rb, Cs, Mg, Ca, Sr, Ba, Sc, Y, La, Ac, Zr, La, Ac, Ce, Th, or a mixture of two or more thereof; a is a number in the range from zero to about 0.5; b is a number in the range from zero to about 0.5; and x is the number of oxygens needed to fulfill the valency requirements of the elements present.

14. The catalyst of claim 1 wherein the catalyst has a cobalt loading level of at least about 10% by weight.

15. The catalyst of claim 1 wherein the catalyst has a cobalt dispersion of at least about 3%.

16. The catalyst of claim 1 wherein the support comprises alumina, and during (A) the support is contacted with cobalt nitrate and perrehenic acid.

17. The catalyst of claim 2 wherein during (B) the supported catalyst is contacted with a mixture of hydrogen and at least one inert gas.

18. The catalyst of claim 1 wherein during (C) the supported catalyst is contacted with a mixture of oxygen and at least one inert gas.

19. The catalyst of claim 1 wherein during (D) the supported catalyst is contacted with a mixture of hydrogen and at least one inert gas.

20. The catalyst of claim 1 wherein during (E) the supported catalyst is contacted with a mixture of oxygen and at least one inert gas.

21. The catalyst of claim 3 wherein during (F) the supported catalyst is contacted with a mixture of hydrogen and at least one inert gas.

22. A process for converting a reactant composition comprising H₂ and CO to a product comprising at least one aliphatic hydrocarbon having at least about 5 carbon atoms, the process comprising:
flowing the reactant composition through a microchannel reactor in contact with the catalyst of claim 1 to convert the reactant composition to the product, the microchannel reactor comprising a plurality of process microchannels containing the catalyst;
transferring heat from the process microchannels to a heat exchanger; and
removing the product from the microchannel reactor.

23. The process of claim 22 wherein each process microchannel has an internal dimension of width or height of up to about 10 mm.

24. The process of claim 22 wherein the process microchannels are made of a material comprising: steel; monel; inconel; aluminum; titanium; nickel; copper; brass; an alloy of any of the foregoing metals; a polymer; ceramics; glass; a composite comprising a polymer and fiberglass; quartz; silicon; or a combination of two or more thereof.

25. The process of claim 22 wherein the heat exchanger comprises heat exchange channels.

26. The process of claim 25 wherein the heat exchange channels comprise microchannels.

27. The process of claim 26 wherein the heat exchange microchannels have internal dimensions of width or height of up to about 10 mm.

28. The process of claim 22 wherein the lengths of the process microchannels and the lengths of the heat exchange channels are about the same.

29. The process of claim 22 wherein the heat exchanger comprises a heat exchange zone adjacent to at least one process microchannel, the heat exchange zone comprising a plurality of heat exchange channels, the heat exchange channels extending lengthwise at right angles relative to the lengthwise direction of the process microchannel, the heat exchange zone extending lengthwise in the same direction as the process microchannel, the length of the heat exchange zone being shorter than the length of the process microchannel, the process microchannel having an entrance and an exit, the heat exchange zone being positioned at or near the process microchannel entrance.

30. The process of claim 22 wherein the heat exchanger comprises two heat exchange zones adjacent to at least one process microchannel, each heat exchange zone comprising a plurality of heat exchange channels, the heat exchange channels extending lengthwise at right angles relative to the lengthwise direction of the process microchannel, the process microchannel having an entrance and an exit, the heat exchange zones extending lengthwise in the same direction as the process microchannel, the lengths of the heat exchange zones being shorter than the length of the process microchannel, the length of one of the heat exchange zones being shorter than the length of the other heat exchange zone, the heat exchange zones being positioned at or near the process microchannel entrance.

31. The process of claim 25 wherein the heat exchange channels are made of a material comprising: steel; monel; inconel; aluminum; titanium; nickel; copper; brass; an alloy of any of the foregoing metals; a polymer; ceramics; glass; a composite comprising polymer and fiberglass; quartz; silicon; or a combination of two or more thereof.

32. The process of claim 22 wherein the microchannel reactor has an entrance and an exit, the product exits the microchannel reactor through the exit, the product being intermixed with unreacted components from the reactant composition, and at least part of the unreacted components from the reactant composition are recycled to the entrance to the microchannel reactor.

33. The process of claim 22 wherein the reactant composition enters the process microchannels and the product exits the process microchannels, the temperature of the reactant composition entering the process microchannels being within about 200° C. of the temperature of the product exiting the process microchannels.

34. The process of claim 22 wherein the mole ratio of H₂ to CO in the reactant composition is in the range of about 0.8 to about 10.

35. The process of claim 22 wherein the reactant composition further comprises H₂O, CO₂, a hydrocarbon of 1 to about 4 carbon atoms, or a mixture of two or more thereof.

36. The process of claim 25 wherein the process microchannels exchange heat with a heat exchange fluid flowing through the heat exchange channels.

37. The process of claim 36 wherein the heat exchange fluid undergoes a phase change as it flows through the heat exchange channels.

38. The process of claim 25 wherein an endothermic process is conducted in the heat exchange channels.

39. The process of claim 38 wherein the endothermic process comprises a steam reforming reaction or a dehydrogenation reaction.

40. The process of claim 25 wherein the reactant composition and product flow through the process microchannels in a first direction, and a heat exchange fluid flow through the heat exchange channels in a second direction, the second direction being cross current relative to the first direction.

41. The process of claim 25 wherein the reactant composition and product flow through the process microchannels flow in a first direction, and a heat exchange fluid flows through the heat exchange channels in a second direction, the second direction being cocurrent relative to the first direction.

42. The process of claim 25 wherein the reactant composition and product flow through the process microchannels in a first direction, and a heat exchange fluid flows through the heat exchange channels in a second direction, the second direction being counter current relative to the first direction.

43. The process of claim 25 wherein a heat exchange fluid flows through the heat exchange channels, the heat exchange fluid comprising air, steam, liquid water, carbon dioxide, gaseous nitrogen, a gaseous hydrocarbon or a liquid hydrocarbon.

44. The catalyst of claim 1 wherein the catalyst is in the form of particulate solids.

45. The process of claim 22 wherein the catalyst is washcoated on interior walls of the process microchannels, grown on interior walls of the process microchannels from solution, or coated in situ on a fin structure.

46. The process of claim 22 wherein the catalyst is supported by a support structure made of a material comprising an alloy comprising Ni, Cr and Fe, or an alloy comprising Fe, Cr, Al and Y.

47. The process of claim 22 wherein the catalyst is supported on a support structure having a flow-by configuration, a flow-through configuration, or a serpentine configuration.

48. The process of claim 22 wherein the catalyst is supported on a support structure having the configuration of a foam, felt, wad, fin, or a combination of two or more thereof.

49. The process of claim 22 wherein the catalyst is supported on a support structure having a flow-by configuration with an adjacent gap, a foam configuration with an adjacent gap, a fin structure with gaps, a washcoat on a substrate, or a gauze configuration with a gap for flow.

50. The process of claim 22 wherein the catalyst is supported on a support structure in the form of a fin assembly comprising at least one fin.

51. The process of claim 50 wherein the fin assembly comprises a plurality of parallel spaced fins.

52. The process of claim 50 wherein the fin has an exterior surface and a porous material overlies at least part of the exterior surface of the fin, the catalyst being supported by the porous material.

53. The process of claim 52 wherein the porous material comprises a coating, fibers, foam or felt.

54. The process of claim 50 wherein the fin has an exterior surface and a plurality fibers or protrusions extend from at least part of the exterior surface of the fin, the catalyst being supported by the protrusions.

55. The process of claim 50 wherein the fin has an exterior surface and the catalyst is: washcoated on at least part of the exterior surface of the fin; grown on at least part of the exterior surface of the fin from solution; or deposited on at least part of the exterior surface of the fin using vapor deposition.

56. The process of claim 50 wherein the fin assembly comprises a plurality of parallel spaced fins, at least one of the fins having a length that is different than the length of the other fins.

57. The process of claim 50 wherein the fin assembly comprises a plurality of parallel spaced fins, at least one of the fins having a height that is different than the height of the other fins.

58. The process of claim 50 wherein the fin has a cross section having the shape of a square, a rectangle, or a trapezoid.

59. The process of claim 50 wherein the fin is made of a material comprising: steel; aluminum; titanium; iron; nickel; platinum; rhodium; copper; chromium; brass; an alloy of any of the foregoing metals; a polymer; ceramics; glass; a composite comprising polymer and fiberglass; quartz; silicon; or a combination of two or more thereof.

60. The process of claim 50 wherein the fin is made of an alloy comprising Ni, Cr and Fe, or an alloy comprising Fe, Cr, Al and Y.

61. The process of claim 50 wherein the fin is made of an Al₂O₃ forming material or a Cr₂O₃ forming material.

62. The process of claim 22 wherein the process microchannels have a bulk flow path comprising about 5% to about 95% of the cross sections of such process microchannels.

63. The process of claim 22 wherein the contact time of the reactant composition and/or product with the catalyst is up to about 2000 milliseconds.

64. The process of claim 22 wherein the temperature of the reactant composition entering the process microchannels is in the range from about 150° C. to about 270° C.

65. The process of claim 22 wherein the pressure within the process microchannels is at least about 5 atmospheres.

66. The process of claim 22 wherein the space velocity for the flow of the reactant composition and product through the process microchannels is at least about 1000 hr.⁻¹

67. The process of claim 22 wherein the pressure drop for the flow of the reactant composition and product through the process microchannels is up to about 10 atmospheres per meter of length of the process microchannels.

68. The process of claim 25 wherein a heat exchange fluid flows through the heat exchange channels, the pressure drop for the heat exchange fluid flowing through the heat exchange channels being up to about 10 atmospheres per meter of length of the heat exchange channels.

69. The process of claim 22 wherein the conversion of CO is about 40% or higher per cycle.

70. The process of claim 22 wherein the yield of product is about 25% or higher per cycle.

71. The process of claim 22 wherein the conversion of CO is at least about 50% per cycle, the selectivity to methane in the product is about 15% or less, and the yield of product is at least about 35% per cycle.

72. The process of claim 22 wherein the catalyst is in the form of particulate solids, the median particle diameter of the particulate solids is in the range of about 1 to about 1000 μm, and the length of each process microchannel is up to about 500 cm.

73. The process of claim 22 wherein the product comprises hydrocarbons boiling at a temperature at or below about 350° C. at atmospheric pressure.

74. The process of claim 22 wherein the product comprises hydrocarbons boiling above a temperature of about 350° C. at atmospheric pressure.

75. The process of claim 22 wherein the product comprises a middle distillate.

76. The process of claim 22 wherein the product comprises at least one olefin.

77. The process of claim 22 wherein the product comprises a normal paraffin, isoparaffin, or mixture thereof.

78. The process of claim 22 wherein the product is further processed using hydrocracking, hydroisomerizing or dewaxing.

79. The process of claim 22 wherein the product is further processed to form a lubricating base oil or a diesel fuel.

80. The process of claim 22 wherein the process microchannels are vertically oriented, the reactant composition and product flow downwardly through the process microchannels.

81. The process of claim 22 wherein the process produces at least about 0.5 gram of aliphatic hydrocarbon having at least about 5 carbon atoms per gram of catalyst per hour; and the selectivity to methane in the product is less than about 25%.

82. The process of claim 22 wherein subsequent to removing the product from the microchannel reactor a regenerating fluid flows through the process microchannels in contact with the catalyst, the residence time for the regenerating fluid in the process microchannels being from about 0.01 to about 1000 seconds.

83. The process of claim 22 wherein the catalyst is regenerated by increasing the molar ratio of H₂ to CO in the reactant composition to at least about 4:1 to provide an adjusted feed composition, and flowing the adjusted feed composition through the process microchannels in contact with the catalyst at a temperature in the range from about 120° C. to about 500° C. for a period of time in the range from about 0.5 to about 48 hours to provide the regenerated catalyst.

84. The process of claim 80 wherein the adjusted feed composition comprises H₂ and is characterized by the absence of CO.

85. The process of claim 80 wherein the process further comprises contacting the regenerated catalyst with the reactant composition comprising H₂ and CO.

86. A process for converting a reactant composition comprising H₂ and CO to a product comprising at least one aliphatic hydrocarbon having at least about 5 carbon atoms, the process comprising contacting the catalyst of claim 1 with the reactant composition to convert the reactant composition to the product.

87. A process for making a catalyst, comprising cobalt and optionally a co-catalyst and/or promoter on a support, the process comprising:
(A) contacting the support with the cobalt and optionally the co-catalyst and/or promoter to form a supported catalyst;
(C) oxidizing the supported catalyst; and
(D) reducing the supported catalyst.

88. The process of claim 87, further comprising subsequent to (A) but prior to (C):
(B) reducing the supported catalyst.

89. The process of claim 87, further comprising subsequent to (D) the following:
(E) oxidizing the supported catalyst; and
(F) reducing the supported catalyst.

90. A process for making a catalyst, comprising cobalt and optionally a co-catalyst and/or promoter on a support, the process comprising:
(A) contacting the support with the cobalt and optionally the co-catalyst and/or promoter to form a supported catalyst;
(B) reducing the supported catalyst;
(C) oxidizing the supported catalyst;
(D) reducing the supported catalyst;
(E) oxidizing the supported catalyst; and
(F) reducing the supported catalyst.

91. The process of claim 87 wherein (A) comprises:
(A-1) contacting the support with a composition comprising at least one cobalt compound and optionally at least one co-catalyst and/or promoter; and
(A-2) calcining the contacted support from (A-1).

92. The process of claim 91 wherein (A) further comprises:
(A-3) contacting the support from (A-2) with another composition comprising at least one cobalt compound and optionally at least one co-catalyst and/or promoter; and
(A-4) calcining the contacted support from (A-3).

93. The process of claim 88 wherein (B) comprises:
(B-1) heating the supported catalyst in the presence of a reducing fluid to a first temperature in the range from about 100° C. to about 500° C. over a period of time in the range from about 0.5 to about 12 hours;
(B-2) maintaining the supported catalyst at the first temperature in the presence of the reducing fluid for a period of time in the range from about 2 to about 25 hours;
(B-3) heating the supported catalyst from (B-2) in the presence of the reducing fluid to a second temperature in the range from about 300° C. to about 600° C. over a period of time in the range from about 0.5 to about 12 hours;
(B-4) maintaining the supported catalyst in the presence of the reducing fluid at the second temperature for a period of time in the range from about 0.5 to about 24 hours; and
(B-5) contacting the supported catalyst from (B-4) with an inert fluid and cooling the supported catalyst to ambient temperature over a period of about 1 to about 48 hours.

94. The process of claim 87 wherein (C) comprises:
(C-1) heating the supported catalyst in the presence of an oxidizing fluid to a first temperature in the range from about 150° C. to about 650° C. over a period of time in the range from about 0.5 to about 12 hours;
(C-2) maintaining the supported catalyst at the first temperature in the presence of the oxidizing fluid for a period of time in the range from about 0.5 to about 10 hours; and
(C-3) contacting the supported catalyst with an inert fluid and cooling the supported catalyst to ambient temperature over a period of time in the range from about 1 to about 48 hours.

95. The process of claim 87 wherein (D) comprises:
(D-1) heating the supported catalyst in the presence of a reducing fluid to a first temperature in the range from about 100° C. to about 500° C. over a period of time in the range from about 0.5 to about 10 hours;
(D-2) maintaining the supported catalyst at the first temperature in the presence of the reducing fluid for a period of time in the range from about 2 to about 25 hours;
(D-3) heating the supported catalyst from (D-2) in the presence of the reducing fluid to a second temperature in the range from about 300° C. to about 600° C. over a period of time in the range from about 0.5 to about 12 hours;
(D-4) maintaining the supported catalyst at the second temperature in the presence of the reducing fluid for a period of time in the range from about 0.5 to about 24 hours; and
(D-5) contacting the supported catalyst with an inert fluid and cooling the supported catalyst to ambient temperature over a period of time in the range from about 1 to about 48 hours.

96. The process of claim 89 wherein (E) comprises:
(E-1) heating the supported catalyst in the presence of an oxidizing fluid to a first temperature in the range from about 150° C. to about 650° C. over a period of time in the range from about 0.5 to about 12 hours;
(E-2) maintaining the supported catalyst at the first temperature in the presence of the oxidizing fluid for a period of time in the range from about 0.5 to about 10 hours; and
(E-3) contacting the supported catalyst with an inert fluid and cooling the supported catalyst to ambient temperature over a period of time in the range from about 1 to about 48 hours.

97. The process of claim 89 wherein (F) comprises:
(F-1) heating the supported catalyst in the presence of a reducing fluid to a first temperature in the range from about 100° C. to about 500° C. over a period of time in the range from about 0.5 to about 20 hours;
(F-2) maintaining the supported catalyst at the first temperature in the presence of the reducing fluid for a period of time in the range from about 0.5 to about 48 hours;
(F-3) heating the supported catalyst from (F-2) in the presence of the reducing fluid to a second temperature in the range from about 100° C. to about 600° C. over a period of time in the range from about 0.5 to about 12 hours;
(F-4) maintaining the supported catalyst in the presence of the reducing fluid at the second temperature for a period of time in the range from about 0.5 to about 48 hours; and
(F-5) contacting the supported catalyst with the reducing fluid at a pressure in the range from about 0.7 to about 75 atm and cooling the supported catalyst to a temperature in the range from about 60° C. to about 250° C. over a period of time in the range from about 0.5 to about 40 hours.

98. The process of claim 87 wherein the catalyst is positioned in a microchannel, step (A) being conducted prior to positioning the catalyst in the microchannel, and steps (C) and (D) being conducted with the catalyst in the microchannel.

99. The process of claim 88 wherein the catalyst is positioned in a microchannel, step (A) being conducted prior to positioning the catalyst in the microchannel, and steps (B), (C) and (D) being conducted with the catalyst in the microchannel.

100. The process of claim 89 wherein the catalyst is positioned in a microchannel, step (A) being conducted prior to positioning the catalyst in the microchannel, and steps (C), (D), (E) and (F) being conducted with the catalyst in the microchannel.

101. The process of claim 90 wherein the catalyst is positioned in a microchannel, step (A) being conducted prior to positioning the catalyst in the microchannel, and steps (B) through (F) being conducted with the catalyst in the microchannel.

102. The process of claim 90 wherein the catalyst is positioned in a microchannel, steps (A) through (E) being conducted prior to positioning the catalyst in the microchannel, and step (F) being conducted with the catalyst in the microchannel.
